(19) United States
(12) Patent Application Publication
(10) Pub. No.: US 2011/0239318 A1
(43) Pub. Date: Sep. 29, 2011

(54) METHODS FOR PRODUCING FISH WITH HIGH LIPID CONTENT

(75) Inventors: David Stephen, Davis, CA (US); Gaye Elizabeth Morgenthaler, Woodside, CA (US)

(73) Assignee: LiveFuels, Inc., San Carlos, CA (US)

(21) Appl. No.: 13/128,858

(22) PCT Filed: Nov. 17, 2009
(86) PCT No.: PCT/US09/64732

§ 371 Date: Jun. 15, 2011

Related U.S. Application Data
(60) Provisional application No. 61/115,607, filed on Nov. 18, 2008.

(51) Int. Cl. A01K 67/027; C12N 15/00; A61K 38/00; C10L 1/02; C10L 1/04; A61P 43/00

(52) U.S. Cl. 800/20; 800/21; 514/10.1; 514/9.9; 514/10.3; 44/385; 585/240

(57) ABSTRACT

The invention provides methods for producing biofuel from algae, that use fish which have a high capacity of producing and/or accumulating lipids to harvest algae from an algal culture. The invention also provides methods for growing fish that result in a high lipid content. The invention also provides methods for creating fish that have a high capacity of producing and accumulating lipids by breeding and/or recombinant DNA techniques. Also included are transgenic fish that have a higher lipid content than wild-type fish.

The application claims the benefit of U.S. Provisional Patent Application Ser. No. 61/115,607, filed Nov. 18, 2008, which is incorporated by reference herein in its entirety.

### 1. INTRODUCTION

The invention relates to methods for producing fish with high lipid content and uses thereof in harvesting algae.

### 2. BACKGROUND OF THE INVENTION

The United States presently consumes about 42 billion gallons per year of diesel for transportation. In 2007, a nascent biodiesel industry produced 250 million gallons of a bio-derived diesel substitute produced from mostly soybean oil in the U.S. It has been proposed to use algae as a feedstock for producing biofuel, such as biodiesel. Some algae strains can produce up to 50% of their dried body weight in triglyceride oils. However, algae currently requires an energy-intensive process to convert algae into an energy feedstock largely because of the significant volume of water that needs to be processed. It has been estimated that on average 20,000 to 40,000 gallons of water need to be processed to recover one gallon of algal oil.

To use algae as an energy crop commercially, the cost of production needs to be significantly reduced. Planktivorous organisms do a cheaper and far more energy-efficient job of dewatering algae and extracting algal components than any method devised thus far by humans. For example, menhaden can filter 7 gallons of water a minute by swimming with their jaws open, and their digestive tracts can process the wide array of inputs (Peck, J. I., 1893. On the food of the menhaden. Bull. U.S. Fish. Comm. 13: 113-126). The use of fish to harvest algae is an energy-efficient and cost-effective method for converting algae into biofuel.

In the past century, humans have learned how to grow certain high-lipid fish species such as salmon. But for the most part, it is the top-of-the-food-chain carnivorous fish that have been “domesticated.” The high-lipid planktivorous fish at the lower end of the food chain—anchovy, herring, menhaden, sardines, shads, etc.—have not been sufficiently valuable to make cultivation economically viable. The use of genetically selected strains and hybrids has contributed very substantially to modern agriculture and animal husbandry. But aquaculture is yet to gain much from breeding and selection programs and transgenic animal technology. To improve the energy efficiency and economics in using planktivorous fish to harvest algae, the present invention provides methods for producing fish with high lipid content that are better suited for harvesting algae than are wild type fish.

### 3. SUMMARY OF THE INVENTION

The invention relates to methods for producing a fish with a high lipid content, and uses of the fish to harvest algae and produce biofuel. In one embodiment, the invention provides methods for producing a fish, comprising the steps of introducing a transgene into a fish, wherein the presence of said transgene results in a modification of the lipid content of said fish, and selecting a fish or a progeny thereof that comprises said transgene, wherein the lipid content of said fish or progeny thereof is greater than the lipid content of a fish without said transgene. The types of lipids present in the fish and their relative abundance are also modified. Depending on whether a stimulator gene or a suppressor gene is involved, the transgene can comprise (i) an expressible stimulator gene; or a gene expression regulatory region that is integrated into the genome and is operably associated with a native stimulator gene such that the stimulator gene is expressed ectopically or constitutively; or (ii) an expressible antisense polynucleotide of a suppressor gene; an expressible polynucleotide that silences expression of a suppressor gene by RNA interference; or a non-expressing allele of a suppressor gene that is integrated into the native suppressor gene in the genome.

The stimulator genes useful in the invention encode, without limitation, neuropeptide Y, pancreatic peptide, agouti-related protein, a secretin, ghrelin, insulin, an insulin-like growth factor, orexin A, orexin B, galanin, a receptor of one of the foregoing factors, PPARγ, lipoprotein lipase, fat-induced transcript 1, or fat-induced transcript 2. The suppressor genes useful in the invention encode, without limitation, leptin, cholecystokinin, cocaine and amphetamine-regulated transcript, corticotropin-releasing factor, bombesin, alpha-melanocyte-stimulating hormone, tachykinin, glucagon-like peptide-1, urotensin I, somatostatin, a receptor of one of the foregoing factors, PPARα, PPARδ, β-glucocerebrosidase, α-galactosidase, β-N-acetylhexosaminidase A, acid sphingomyelinases, NPC1, or NPC2. Uses of homologs or orthologs of these stimulator and suppressor genes are contemplated. The invention also contemplates creating a model of fish with high lipid content using stimulator or suppressor genes as a transgene in zebrafish.

In another embodiment, the invention provides methods for producing a fish, comprising the steps of reproducing a population of fish according to a breeding program that is directed to modifying a phenotype, wherein the phenotype is lipid content, and selecting a fish from a succeeding generation in the breeding program, wherein the lipid content of said fish is greater than the lipid content of fish of an earlier generation. A second phenotype such as growth rate can also be selected in the breeding program. A breeding program can comprise various steps including at least one of inbreeding, selective breeding, crossbreeding, induction of polyploidy, gynogenesis or androgenesis.

To tailor a specialized fish for harvesting algae, the selecting steps of the invention can comprise feeding the fish with algae from an algal culture of defined composition for a period of time, prior to determining the lipid content of said fish. The lipid content of fish can optionally be estimated by determining the moisture content of the fish or a part or an organ of the fish.

In yet another embodiment, the invention provides methods for culturing fish, comprising administering an antagonist of a fish hormone to a fish to prevent sexual maturation of the fish, wherein the fish hormone is lutenizing hormone, follicle stimulating hormone, or gonadotropin releasing hormone, and wherein the growth rate of a sexually mature fish is lower than the growth rate of a sexually immature fish, thereby increasing the lipid content of the fish.

In yet another embodiment, the invention provides methods for culturing fish, comprising administering a fish hormone or an agonist thereof to a fish to accelerate sexual maturation of a female fish, wherein the fish hormone is lutenizing hormone, follicle stimulating hormone, or gonadotropin releasing hormone, or an analog thereof, and wherein the lipid content of a sexually mature female fish is greater than the lipid content of a sexually immature female fish. In certain embodiments, it is desirable to use a monosex fish population to harvest algae. Hormones or an agonist thereof, such as estradiol-17β oestrone, oestriol, diethylstilbestrol, diethylstilbestrol diphosphate, diethylstilbestrol dipropionate, or 17α-ethyyloestradiol, can be used to create a monosex population of fish. In a specific embodiment, a monosex female fish population is produced to harvest algae. The culture methods of the invention generally comprise feeding the fish with algae from an algal culture, wherein the composition of the culture or the proportion of different algae species in the culture is defined.

Also encompassed are the fish produced or cultured by the methods of the invention, including a fish comprising a transgene wherein the presence of said transgene results in a lipid content higher than a fish without said transgene; a fish produced by a breeding program that is directed to modifying the lipid content of fish which results in a lipid content that is greater than the lipid content of the parental fish. In certain embodiments, the quality of lipids of the fish comprising the transgene is also modified. In various embodiments, the fish used in the methods of the invention are preferably planktivores or omnivores. Preferably, the fish are members of Clupiformes. In specific embodiments, the fish is a menhaden, shad, herring, sardine, hilsa, anchovy, milkfish, catfish, barb, carp, zebrafish, goldfish, loach, shiner, minnow, rasbora, Labeo species, smelt, or mullet.

In another embodiment, the invention provides methods for producing biofuel, comprising the steps of using a fish created or cultured by the methods of the invention to harvest algae, extracting oil from the fish, and converting the oil to biofuel. Beside using the fish to make biofuel, the fish or parts thereof can also be used as human food, as a source of highly unsaturated fatty acids useful as a nutritional supplement, as an industrial feedstock for making various oleochemical-derived products, and as agricultural and/or aquaculture feed.

### 4. DETAILED DESCRIPTION OF THE INVENTION

Algal biomass rich in lipids is a source of energy and industrial feedstocks, as well as food. Many fishes feed on algae and store the energy as lipids. Fishes can recover some of the energy and biomass lost to zooplanktons that graze on phytoplanktons, or in detritus. Gathering farmed fishes is less energy intensive than harvesting algae from a large body of water. Instead of harvesting algae and extracting lipids from the algae, fishes that feed on algae can be used to harvest the algae effectively and efficiently. Oil extracted from the fishes can be used as feedstock for making biofuel. The present invention makes the algae-harvesting process even more energy efficient by using specialized fishes and culture methods. For the same investments in farming/processing infrastructure and energy expenditure, the invention results in a greater yield of lipids.

The invention provides methods for culturing fishes that result in fishes with a high lipid content. The invention also provides the creation of genetically improved fish that have a high capacity of producing and/or accumulating lipids. Also encompassed are methods of making biofuels from the fishes with a high lipid content. Depending on the species, fishes of the invention with a high lipid content can also be used for human consumption, making animal feed including aquaculture feed, and making a variety of other oleochemical-derived products, such as paints, linoleum, lubricants, soaps, insecticides, and cosmetics. The fish is also a source of highly unsaturated fatty acids, such as a-linolenic acid (ALA), eicosapentaenoic acid (EPA), and docosahexaenoic acid (DHA), that can be used in manufacturing nutritional supplements.

The inventors seek to improve the harvesting process by taking two approaches: (i) fish culturing methods including the use of biologics such as hormones; and (ii) using genetically improved fishes that are predisposed to have a high lipid content. The approaches can be applied separately or in combination. In various embodiments, the methods of the invention comprises using a fish to harvest algae wherein the lipid content of the fish or a part thereof is higher than a control fish. According to the invention, a fish that has a high lipid content can be obtained by creating a genetically improved line of fish and/or by applying fish culturing methods of the invention. The fishes of the invention are expected to have a lipid content that is at least 0.5%, 1%, 2%, 3%, 4%, 5%, 6%, 7%, 8%, 9%, 10%, 11%, 12%, 13%, 14%, 15%, 20%, 25%, 30%, 40%, or 50% higher than a control fish. In certain embodiments of the invention, both the quantity and quality of the lipids are modified, for example, the relative abundance of various lipids are changed and/or new species or types of lipids are produced, in the improved fish.

The term “improved fish” or “genetically improved fish” refers to a fish that is genetically predisposed to having a lipid content that is higher than the control fish, when they are cultured under the same conditions. The improved fish possesses a higher capacity of producing and/or accumulating lipids on a diet of algae. The control fish is a fish of the same gender at a comparable age at the time of the experiment. It can be a wild type fish, a fish of the same breed that is captured from the wild or cultured by conventional methods under the same environmental conditions, or a reference species. The control fish can be the unimproved parent of a genetically improved fish. Genetically improved fish can be created by breeding and/or by recombinant DNA technology. The population of fishes that is used to harvest algae can comprise a single species of fish, multiple species, as well as a mix of wild type and genetically improved fishes. Most culture techniques of the invention such as the use of biologics, can be applied to a mixed population of fishes. However, methods for improving the genetic constitution of fish are preferably applied to one species of fish at a time. The starting fish population can be acquired from a hatchery or from the wild where it has similar environmental conditions as the harvesting operation. For example, an endemic fish population can be used. The fishes that can be cultured or improved by the methods of the invention are described in section 4.1.

In one embodiment of the invention, fish breeding programs are established to produce a genetically improved fish. Fish breeding programs based on inbreeding, selective breeding, crossbreeding, chromosomal manipulations, or a combination of the foregoing, can be used. The offspring in a breeding program are selected for high lipid content. The selection methods of the invention involves feeding the fish with an algal composition for a period of time, and measuring the lipid content. Detailed descriptions of the selection and breeding methods of the invention are provided in sections 4.2 and 4.5, respectively.

In another embodiment, the invention comprise engineering targeted changes to the genetic information of a fish. The inventors recognize that there are generally two types of genes that affect lipid content: stimulator genes—the expression of which is correlated with a higher lipid content, and suppressor genes—the expression of which is correlated with a lower lipid content. Collectively, the stimulator genes and suppressor genes are referred to as “target genes.” A number of genetic engineering strategies are contemplated for producing a transgenic fish that has a high capacity for producing and/or accumulating lipids. The invention provides a transgenic fish in which the expression of a stimulator gene is increased thereby increasing the lipid content of the fish. The expression of stimulator gene can be increased by one of several techniques known in the art, such as but not limited to, increasing the copy number of the stimulator gene, introducing a homolog of the stimulator gene, overexpressing the stimulator gene, or deregulating expression of the stimulator gene. In a specific embodiment, the transgene comprises an expressible stimulator gene. In another embodiment, the invention provides a transgenic fish wherein the expression of a suppressor gene is decreased, thereby increasing the lipid content of the fish. The decrease of suppressor gene expression can be accomplished by techniques well known in the art, such as but not limited to, knocking out the suppressor gene in the fish genome or use of antisense nucleotides, including RNA interference, to knockdown suppressor gene expression. Detailed description of the strategies and recombinant DNA constructs that are used in these strategies are provided in section 4.3.

While the stimulator and suppressor genes are engineered in transgenic fishes of the invention resulting in the observable phenotype of high lipid content, referred to herein as “obese,” it is not necessary to know the mechanisms of physiologic action of these genes. However, without being bound by any particular theory, the target genes useful in the invention play a role in energy homeostasis, appetite regulation, lipid transport and metabolism, adipose tissue development, and human diseases related to obesity, lipid metabolism, and diabetes.

In one embodiment, the target genes of the invention are involved in appetite regulation, such as but not limited to genes encoding hypothalamic neuropeptides. The hypothalamus integrates input from factors that stimulate (orexigenic) and inhibit (anorexigenic) food intake. In teleost fish, the identification of appetite regulators has been achieved by the use of both peptide injections followed by measurements of food intake, and by molecular cloning combined with gene expression studies. Accordingly, genes encoding orexigenic factors can be used as stimulator genes and genes encoding anorexigenic factors can be used as suppressor genes. Neuropeptide Y (NPY) is one of most potent orexigenic factors in fish. Other orexigenic factors include but are not limited to pancreatic peptide (PP), agouti-related protein (AgRP), secretins, and ghrelin (secreted in stomach), orexin A and B and galanin. The latter three factors have been found to interact with NPY in the control of food intake in an interdependent and coordinated manner. Anorexigenic factors include but are not limited to leptin, cholecystokinin (CCK), cocaine and amphetamine-regulated transcript (CART), corticotropin-releasing factor (CRF), bombesin (or gastrin-releasing peptide), alpha-melanocyte-stimulating hormone (alpha-MSH), tachykinins, glucagon-like peptide-1 (GLP-1) and urotensin I. In addition, the use of genes encoding receptors of these endocrine factors, such as neuropeptide Y receptor, melanocortin receptor 4 (MC4-R), are contemplated. A full discussion of the biology underlying appetite regulation in fish is provided in “Neuropeptides and the control of food intake in fish” by Volkoff H, et al. Gen Comp Endocrinol. 2005, 142(1-2):3-19; and Metz et al., Gen Comp Endocrinol. (2006) 148(2):150-62.

In another embodiment, the stimulator and suppressor genes are involved in energy homeostasis. Insulin facilitates assimilation by promoting the uptake of nutrient molecules (e.g., glucose, amino acids, and fatty acids) into cells. Glucose transporter proteins (GLUT) mediate the diffusion of glucose into skeletal muscle cells. Insulin and insulin-like growth factors (e.g., IGF-1 and IGF-2) are generally anabolic and stimulates the synthesis and deposition of energy reserves (e.g., glycogen, triacylglycerol) as well as of proteins, thereby facilitating organismal growth. Insulin favors lipogenesis and glycogenesis by reducing plasma lipid levels and increasing stored lipids in adipose tissue and liver. Breakdown and mobilization of stored energy reserves is stimulated by catabolic factors, such as glucagon, GLP-1, and somatostatin. Somatostatins stimulate the breakdown of stored triacylglycerols and glycogen in storage tissues. The use of genes encoding receptors of these anabolic and catabolic hormones are also contemplated. Genes that play a role in lipogenesis are thus stimulator genes of the invention while genes that promote lipolysis are suppressor genes of the invention, e.g., lipoprotein lipase. A discussion of the biology underlying gastrointestinal hormones and metabolism in fish is provided in Nelson and Sheridan, 2006, Gen. Comp. Endocrinol. 148:116-124.

In yet another embodiment, genes encoding targets for finding drugs to treat obesity, diabetes, and hyperlipidemia in human can also be used as stimulator and suppressor genes of the invention. For example, peroxisome proliferators-activator receptors (PPARγ, PPARδ) are sensitive to levels of fatty acids and cause transcriptional changes that alter the utilization of lipids and glucose. Thiazolidinediones are PPARγ agonists used for treating type II diabetes which cause weight gain in humans through adipogenesis. Thus, the PPARγ gene can be used as a stimulator gene of the invention. On the other hand, suppression of expression of PPARα or PPARδ (also known as PPARβ) in mice was observed to lead to obesity. Thus, the PPARα and PPARβ genes can be used as a suppressor gene in the invention.

In yet another embodiment, genes encoding enzymes underlying human diseases associated with lipid metabolism can be used as stimulator and suppressor genes of the invention. For example, genes encoding β-glucocerebrosidase (Gaucher's disease), α-galactosidase (Fabry's disease), β-N-acetylhexosaminidase A (Tay-Sachs disease), acid sphingomyelinases (Niemann-Pick diseases A and B), and NPC1 and NPC2 genes involved in cholesterol transport and cholesterol accumulation (Niemann-Pick disease C), can be used as suppressor genes of the invention.

A list of exemplary stimulator and suppressor genes are provided in section 4.3. Although many stimulator and suppressor genes are known in the art, some of the orthologous stimulator and suppressor genes in the species of fish that is to be improved, may not be cloned. The invention contemplates using a functionally homologous or orthologous gene as the transgene in fish. The invention also contemplates isolating the stimulator and suppressor genes from the fish species of interest and using the isolated gene for genetic improvement of that species. Techniques for isolating homologous gene sequences from another species by hybridization and/or polymerase chain reaction are well known in the art, and are described in section 4.3.

The invention also provides the use of the zebrafish genetic system to model the effects of stimulator and suppressor genes. Zebrafish (Danio rerio) belongs to the minnow family, Cyprinidae, and is a close relative of minnows and carps. Use of the model system accelerates the development process and helps prioritize the gene(s) that are to be used as a transgene. As the sequencing of the zebrafish genome reaches completion, it is becoming clear that there is a high degree of genetic conservation between man and fish despite million years of divergent evolution. For example, after a comparison of the endocrine system of zebrafish to those of human and mouse, it was deemed sufficiently similar to serve as a model to study the endocrine system (2006, McGonnell and Fowkes. “Fishing for gene function—endocrine modelling in the zebrafish,” J Endocrinol. 189(3):425-39). Moreover, lipid transport and lipolysis in fish is similar to that observed in mammal with slightly different absorptional and depositional processes (1988, Sheridan, Lipid dynamics in fish: Aspects of absorption, transportation, deposition and mobilization. Comp. Biochem. Physiol. B. 90:679-690). To exploit the knowledge on human and mouse genes that plays a role in obesity and metabolic diseases, the inventors contemplate using zebrafish to examine how these human and mouse genes and their fish homologs affect lipid content, with a view towards using these human and mouse genes and their fish homologs as transgenes to increase the lipid content and modify the lipid quality of fish. This aspect of the invention is a reversal of the direction of inquiry, starting with genes that are already known to cause human diseases, which are then used as transgenes in zebrafish to model obesity that is desired in another fish species. Accordingly, the methods of the invention comprise modulating the expression of a target gene or a transgene in a transgenic zebrafish, measuring the lipid content of the transgenic zebrafish or a part thereof, and producing a transgenic fish (which is not necessarily a zebrafish) with the stimulator or suppressor transgene or a homolog thereof. Methods of using lipid dyes, labeled lipids, and fluorescent reporters to assess the lipid content of zebrafish and its larvae are well known in the art and are used in the methods of the invention.

The invention also provides the use of a defined algal composition to select, identify and characterize genetically improved fish. Since the improved fish of the invention are used for harvesting algae, the selection methods use a particular species of algae or a mixed population of algae to feed the fish. Preferably, the algae used in the selection process are the algae that will be harvested by the fish. It is contemplated that a defined algal composition can be prepared by mixing different algae from a plurality of algal cultures in specific proportions. The algae that are used or harvested in the methods of the invention are described in Section 4.5.

In yet another embodiment, the invention also provides methods of culturing fish that involves administering hormone(s) to modulate the time of sexual maturation. Such methods can be applied to culturing genetically improved fish. A detailed description of the culture methods of the invention is provided in section 4.7.

The fish with high lipid content are gathered and processed by methods known in the art to produce fish oil and fish meal. The technology for lipid extraction and biofuel manufacturing is described in section 4.8. Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full. The practice of the invention will employ, unless otherwise indicated, techniques of chemistry, biology, and the aquaculture industry, which are within the skill of the art. Such techniques are explained fully in the literature, e.g., Aquaculture Engineering, Odd-Ivar Lekang, 2007, Blackwell Publishing Ltd.; Handbook of Microalgal Culture, edited by Amos Richmond, 2004, Blackwell Science; Aquaculture Genome Technologies, by Zhanjiang Liu, Blackwell Pub., 2007; The Laboratory Fish (Handbook of Experimental Animals) by Gary Ostrander (Author), Gillian R. Bullock (Series Editor), Tracie Bunton (Series Editor) 2000 Academic Press; Zebrafish: A Practical Approach (The Practical Approach Series, 261) by Christiane Nusslein-Volhard and Ralf Dahm (Editors) Oxford University Press; Sambrook, Fritsch, and Maniatis, Molecular Cloning; Laboratory Manual 2nd ed. (1989); DNA Cloning, Volumes I and II (D. N. Glover ed. 1985); Oligonucleotide Synthesis (M. J. Gait ed, 1984); Nucleic Acid Hybridization (B. D. Hames & S. J. Higgins eds. 1984); the series, Methods in Enzymology (Academic Press, Inc.), particularly Vol. 154 and Vol. 155 (Wu and Grossman, eds.); PCR—A Practical Approach (McPherson, Quirke, and Taylor, eds., 1991); Oligonucleotide Synthesis, 1984, (M. L. Gait ed); Transcription and Translation, 1984 (Hames and Higgins eds.); Martin J. Bishop, ed., Guide to Human Genome Computing, 2d Edition, Academic Press, San Diego, Calif. (1998); and Leonard F. Peruski, Jr., and Anne Harwood Peruski, The Internet and the New Biology: Tools for Genomic and Molecular Research, American Society for Microbiology, Washington, D.C. (1997), each of which are incorporated by reference in their entireties.

As used herein, “a” or “an” means at least one, unless clearly indicated otherwise. The term “about,” as used herein, unless otherwise indicated, refers to a value that is no more than 20% above or below the value being modified by the term. For clarity of disclosure, and not by way of limitation, the detailed description of the invention is divided into the subsections which follow.

### 4.1 Fishes

As used herein, the term fish refers to a member or a group of the following classes: Actinopteryii (i.e., ray-finned fish) which includes the division Teleosteri (also known as the teleosts), Chondrichytes (e.g., cartilaginous fish), Myxini (e.g., hagfish), Cephalospidomorphi (e.g., lampreys), and Sarcopteryii (e.g., coelacanths). The teleosts comprise at least 38 orders, 426 families, and 4064 genera. Some teleost families are large, such as Cyprinidae, Gobiidae, Cichlidae, Characidae, Loricariidae, Balitoridae, Serranidae, Labridae, and Scorpaenidae. In many embodiments, the invention involves bony fishes, such as the teleosts, and/or cartilaginous fishes. When referring to a plurality of organisms, the term “fish” is used interchangeably with the term “fishes” regardless of whether one or more than one species are present, unless clearly indicated otherwise. Fishes useful for the invention can be obtained from fish hatcheries or collected from the wild. The fishes may be fish fry, juveniles, fingerlings, or adult/mature fish. By “fry” it is meant a recently hatched fish that has fully absorbed its yolk sac, while by “juvenile” or “fingerling” it is meant a fish that has not recently hatched but is not yet an adult. In certain embodiments of the invention, fry and/or juveniles can be used. Any fish aquaculture techniques known in the art can be used to stock, maintain, reproduce, and gather the fishes used in the invention.

One or more species of fish can be used to harvest the algae in an algal composition. A fish of the invention can be produced by a method that comprises (i) reproducing a population of fish according to a breeding program that is directed to modifying a phenotype, wherein said phenotype is lipid content or lipid content and quality, and (ii) selecting a fish from a succeeding generation in the breeding program, wherein the lipid content of said fish is greater than the lipid content of fish of an earlier generation. In one embodiment of the invention, the population of fish comprises only genetically improved fish. In another embodiment, the fish population is mixed and thus comprises one or several major species of fish including genetically improved fish. A major species is one that ranks high in the head count, e.g., the top one to five species with the highest head count relative to other species. In a preferred embodiment, at least one breed of genetically improved fish, considered a species in this context, is a major species in the population. The one or several major fish species may constitute greater than about 10%, about 20%, about 30%, about 40%, about 50%, about 60%, about 70%, about 75%, about 80%, about 90%, about 95%, about 97%, about 98% of the fish present in the population. In certain embodiments, several major fish species may each constitute greater than about 10%, about 20%, about 30%, about 40%, about 50%, about 60%, about 70%, or about 80% of the fish present in the population. In various embodiments, one, two, three, four, five major species of fish are present in a population. Accordingly, a mixed fish population or culture can be described and distinguished from other populations or cultures by the major species of fish present. The fish population or culture can be further described by the percentages of the major and minor species or the breed(s) of genetically improved fishes, or the percentages of each of the major species. It is to be understood that mixed cultures having the same genus or species may be different by virtue of the relative abundance of the various genus and/or species present.

Fish inhabit most types of aquatic environment, including but not limited to freshwater, brackish, marine, and briny environments. As the present invention can be practiced in any of such aquatic environments, any freshwater species, stenohaline species, euryhaline species, marine species, species that grow in brine, and/or species that thrive in varying and/or intermediate salinities, can be used. Fishes from tropical, subtropical, temperate, polar, and/or other climatic regions can be used. Fishes that live within the following temperature ranges can be used: below 10° C., 9° C. to 18° C., 15° C. to 25° C., 20° C. to 32° C. In one embodiment, fishes indigenous to the region at which the methods of the invention are practiced, are used. Preferably, fishes from the same climatic region, same salinity environment, or same ecosystem, as the algae are used. Most preferably, the algae and the fishes are derived from a naturally occurring trophic system.

In an aquatic ecosystem, fish occupies various trophic levels. Depending on diet, fish are classified generally as piscivores (carnivores), herbivores, planktivores, detritivores, and omnivores. The classification is based on observing the major types of food consumed by fish and its related adaptation to the diet. For example, many species of planktivores develop specialized anatomical structures to enable filter feeding, e.g., gill rakers and gill lamellae. Generally, the size of such filtering structures relative to the dimensions of plankton, including microalgae, affects the diet of a planktivore. Fish having more closing spaced gill rakers with specialized secondary structures to form a sieve are typically phytoplanktivores. Others having widely spaced gill rakers with secondary barbs are generally zooplanktivores. In the case of piscivores, the gill rakers are generally reduced to barbs. Herbivores generally feed on macroalgae and other aquatic vascular plants. Gut content analysis can determine the diet of an organism used in the invention. Techniques for analysis of gut content of fish are known in the art. As used herein, a planktivore is a phytoplanktivore if a population of the planktivore, reared in water with non-limiting quantities of phytoplankton and zooplankton, has on average more phytoplankton than zooplankton in the gut, for example, greater than 50%, 60%, 70%, 80%, or 90%. Under similar conditions, a planktivore is a zooplantivore if the population of the planktivore has on average more zooplankton than phytoplankton in the gut, for example, greater than 50%, 60%, 70%, 80%, or 90%. Certain fish can consume a broad range of food or can adapt to a diet offered by the environment. Accordingly, it is preferable that the fish are cultured in a system of the invention before undergoing a gut content.

Fishes that are used in the methods of the invention feed on algae, but it is not required that they feed exclusively on microalgae, i.e., they can be herbivores, omnivores, planktivores, phytoplanktivores, zooplanktivores, or generally filter feeders, including pelagic filter feeders and benthic filter feeders. In certain embodiments of the invention, the fishes used in the invention are planktivores, including but not limited to obligate planktivores. In other embodiments, the fishes are omnivores. In certain embodiments, one or several major species are phytoplanktivores. In other embodiments, one or several species are zooplanktivores. In certain mixed fish population of the invention, planktivores and omnivores are both present. In addition to planktivores, omnivores, herbivores and/or detritivores can also be used in the methods of the invention.

Fishes from different taxonomic groups can be used in the methods of the invention. It should be understood that, in various embodiments, fishes within a taxonomic group, such as a family or a genus, can be used interchangeably in various methods of the invention. The invention is described below using common names of fish groups and fishes, as well as the scientific names of exemplary species. Databases, such as FishBase by Froese, R. and D. Pauly (Ed.), World Wide Web electronic publication, www.fishbase.org, version (06/2008), provide additional useful fish species within each of the taxonomic groups that are useful in the invention. It is contemplated that one of ordinary skill in the art could, consistent with the scope of the present invention, use the databases to specify other species within each of the described taxonomic groups for use in the methods of the invention.

In certain embodiments of the invention, the fishes used in the invention are the order Acipeneriformes, such as but not limited to, sturgeons (trophic level 3) e.g., Acipenser species, Huso huso, and paddlefishes (plankton-feeder), e.g., Psephurus gladius, Polyodon spathula, and Pseudamia zonata.

In certain embodiments of the invention, the fishes used in the invention are in the order Clupiformes which include the following families: Chirocentridae, Clupeidae (menhadens, shads, herrings, sardines, hilsa), Denticipitidae, and Engraulidae (anchovies). Exemplary members within the order Clupiformes include but are not limited to, the menhadens (Brevoortia species), e.g, Ethmidium maculatum, Brevoortia aurea, Brevoortia gunteri, Brevoortia smithi, Brevoortia pectinata, Gulf menhaden (Brevoortia patronus), and Atlantic menhaden (Brevoortia tyrannus); the shads, e.g., Alosa alosa, Alosa alabamae, Alosa fallax, Alosa mediocris, Alosa sapidissima, Alosa pseudoharengus, Alosa chrysochloris, Dorosoma petenense; the herrings, e.g., Etrumeus teres, Harengula thrissina, Pacific herring (Clupea pallasii pallasii), Alosa aestivalis, Ilisha africana, Ilisha elongata, Ilisha megaloptera, Ilisha melastoma, Ilisha pristigastroides, Pellona ditchela, Opisthopterus tardoore, Nematalosa come, Alosa aestivalis, Alosa chrysochloris, freshwater herring (Alosa pseudoharengus), Arripis georgianus, Alosa chrysochloris, Opisthonema libertate, Opisthonema oglinum, Atlantic herring (Clupea harengus), Baltic herring (Clupea harengus membras); the sardines, e.g., Ilisha species, Sardinella species, Amblygaster species, Opisthopterus equatorialis, Sardinella aurita, Pacific sardine (Sardinops sagax), Harengula clupeola, Harengula humeralis, Harengula thrissina, Harengula jaguana, Sardinella albella, Sardinella janeiro, Sardinella fimbriata, oil sardine (Sardinella longiceps), and European pilchard (Sardina pilchardus); the hilsas, e.g., Tenuolosa species, and the anchovies, e.g., Anchoa species, Engraulis species, Thryssa species, anchoveta (Engraulis ringens), European anchovy (Engraulis encrasicolus), Australian anchovy (Engraulis australis), and Setipinna phasa, Coilia dussumieri.

In certain embodiments of the invention, the fishes used in the invention are in the superorder Ostariophysi which include the order Gonorynchiformes, order Siluriformes, and order Cypriniformes. Non-limiting examples of fishes in this group include milkfishes, catfishes, barbs, carps, danios, zebrafish, goldfishes, loaches, shiners, minnows, and rasboras. Milkfishes, such as Chanos chanos, are plankton feeders. The catfishes, such as channel catfish (Ictalurus punctatus), blue catfish (Ictalurus furcatus), catfish hybrid (Clarias macrocephalus), Ictalurus pricei, Pylodictis olivaris, Brachyplatystoma vaillantii, Pinirampus pirinampu, Pseudoplatystoma tigrinum, Zungaro zungaro, Platynematichthys notatus, Ameiurus catus, Ameiurus melas are detritivores. Carps are freshwater herbivores, plankton and detritus feeders, e.g., common carp (Cyprinus carpio), Chinese carp (Cirrhinus chinensis), black carp (Mylopharyngodon piceus), silver carp (Hypophthalmichthys molitrix), bighead carp (Aristichthys nobilis) and grass carp (Ctenopharyngodon idella). Shiners include members of Luxilus, Cyprinella and Notropis genus, such as but not limited to, Luxilus cornutus, Notropis jemezanus, Cyprinella callistia. Other useful herbivores, plankton and detritus feeders are members of the Labeo genus, such as but not limited to, Labeo angra, Labeo ariza, Labeo bata, Labeo boga, Labeo boggut, Labeo porcellus, Labeo kawrus, Labeo potail, Labeo calbasu, Labeo gonius, Labeo pangusia, and Labeo caeruleus.

In certain embodiments of the invention, the fishes used in the invention are in the superorder Protacanthopterygii which include the order Salmoniformes and order Osmeriformes. Non-limiting examples of fishes in this group include the salmons, e.g., Oncorhynchus species, Salmo species, Arripis species, Brycon species, Eleutheronema tetradactylum, Atlantic salmon (Salmo salar), red salmon (Oncorhynchus nerka), and Coho salmon (Oncorhynchus kisutch); and the trouts, e.g., Oncorhynchus species, Salvelinus species, Cynoscion species, cutthroat trout (Oncorhynchus clarkii), and rainbow trout (Oncorhynchus mykiss); which are trophic level 3 carnivorous fish. Other non-limiting examples include the smelts and galaxiids (Galaxia species). Smelts are planktivores, for example, Spirinchus species, Osmerus species, Hypomesus species, Bathylagus species, Retropinna retropinna, and European smelt (Osmerus eperlanus).

In certain embodiments of the invention, the fishes used in the invention are in the superorder Acanthopterygii which include the order Mugiliformes, Pleuronectiformes, and Perciformes. Non-limiting examples of this group are the mullets, e.g., striped grey mullet (Mugil cephalus), which include plankton feeders, detritus feeders and benthic algae feeders; flatfishes which are carnivorous; the anabantids; the centrarchids (e.g., bass and sunfish); the cichlids, the gobies, the gouramis, mackerels, perches, scats, whiting, snappers, groupers, barramundi, drums, wrasses, and tilapias (Oreochromis sp.). Examples of tilapias include but are not limited to nile tilapia (Oreochromis niloticus), red tilapia (O. mossambicus×O. urolepis hornorum), and mango tilapia (Sarotherodon galilaeus).

### 4.2 Breeding Methods

In one embodiment of the invention, the genetically improved fish can be produced by breeding. As used herein the term “breeding” encompasses any reproductive methods that result in a heritable change in the genetic constitution of a lineage of fish. Such reproductive methods include matings, artificial fertilization, and chromosomal manipulation (such as gynogenesis, androgenesis, and polyploidy), but exclude the use of recombinant DNA technologies which is described in section 4.3. Applicable breeding programs include inbreeding, selective breeding, and crossbreeding.

Generally, the invention encompasses methods for producing an improved fish, comprising reproducing a population of fish according to a breeding program, selecting from the offspring an improved fish that has a higher lipid content than at least one of the parent fish or a fish of an earlier generation, or the mean lipid content of the initial fish population or the fish in an earlier generation. The invention also encompasses the improved fish, its gametes (sperms and eggs), embryos, and progeny. As used herein, a progeny of a fish is a fish descended from the first fish by sexual reproduction or cloning, and from which genetic material has been inherited.

The phenotype that is bred into the fishes in the breeding programs of the invention is high lipid content. A surrogate phenotype can also be used if a correlation between high lipid content and the surrogate phenotype is detected. However, the breeding programs can be expanded to include other secondary phenotype(s), such as growth rate, body length, body conformation, resistance to particular diseases, reproductive ability at lower temperature than natural habitat of a parent, and delaying maturation to prevent early switch of metabolism to develop sexual functions. For example, improving body conformation can increase yields as a thicker-bodied fish will carry more muscle on its frame per centimeter body length than a streamlined fish. Methods for selecting the desired offspring by measurement of lipid content and/or surrogate phenotypes(s) are described in section 4.4.

Different breeding programs can be combined or used in tandem to produce the improved fish of the invention. Inbreeding is the mating of relatives or fish more closely related than the population average, resulting in inbred offspring. Crossbreeding is the mating of individuals less closely related than the population average, resulting in hybrid offspring. Selective breeding involves comparing the phenotype's mean of a population over time to an unselected control population, and allowing the superior individuals to mate. Chromosomal manipulations are applicable during nuclear cycles of cell division, and can include but are not limited to, the induction of polyploidy (triploidy, tetraploidy, e.g., triploid carp), gynogenesis (e.g., in catfish), or androgenesis (fish with all paternal genetic materials, e.g., in rainbow trout and in carps), in either gametes before fertilization, or to the fertilized egg. Gynogenesis is a type of parthenogenesis wherein an egg is stimulated to divide by a genetically inactive spermatozoon resulting in a fish with all maternal genetic material.

A transgenic fish as described in section 4.3 can also be used in mating with other transgenic fish or non-transgenic fish in the breeding programs of the invention. Fish hatchery practices and breeding programs well known in the art can be applied. See, for example, Gjedrem, T. 2005, “Selection And Breeding Programs In Aquaculture,” Springer; Tave D, 1999, “Inbreeding and Brood Stock Management,” Fisheries Technical Paper 392, FAO United Nations; Tave D, 1995, “Selective Breeding Programmes,” Fisheries Technical Paper 352, FAO United Nations; Purdom, Colin, 1993, “Genetics and Fish Breeding,” Kluwer; Tave D. 1993, “Genetics for fish hatchery managers,” 2^{nd} ed., Van Nostrand Reinhold, New York; and Kirpichnikov VS, 1981, “Genetic Bases of Fish Selection,” Springer-Verlag, New York; Arai K. “Genetic improvement of finfish species by chromosomal manipulation techniques in Japan,” Aquaculture 197, issues 1-4:205-228, 2001; Khan, T. A., Bhise, M. P. and Lakra, W. S. “Chromosome manipulations in fish—a review.” Indian Journal of Animal Sciences 70: 213-221, 2000; Pandian, T. J. and Koteeswaran, R. “Ploidy induction and sex control in fish,” Hydrobiologia 384: 167-243, 1998.

In one embodiment, the methods of the invention comprise an inbreeding program. Any known inbreeding techniques or programs for producing a new breed or variety can be used. In this method, when a male is considered to be superior in lipid content to all others in a population, that male is bred to many females and a number of his daughters and grand-daughters in order to produce a population of fish that resembles him in lipid content. For example, a male fish is allowed to mate and its offspring and second generation offspring are allowed to mate with a member of the population; then the male fish is brought back to mate with its great-grand child. Another example of inbreeding involves mating a male individual repeatedly to his daughter, grand-daughter, great-grand daughter, etc. The latter program can produce individuals that are genetically very similar to the male. Other matings useful in an inbreeding program include but are not limited to, parent-offspring, brother-sister (full sibs), half brother-half sister(half sibs), grandparent-grandchild, aunt-nephew or uncle-niece, first cousins, second cousins, and double first cousins (first cousins that are twice as related as regular first cousins because the parents that produced them are a pair of full sibs that mated with another pair of full sibs). An inbreeding program of the invention comprises at least one of the matings described above. The resulting inbred offspring can be maintained as a new variety of genetically improved fish. In a specific embodiment, two different inbred lines of fish can be crossbred to produce hybrids with both superior traits.

In another embodiment of the invention, the methods comprise a selective breeding program. Selection procedures can operate at the individual level or at the family level, where whole families are selected or culled based on family means (i.e., between-family selection) or; where the best fish from each of a number of families are saved (i.e., within-family selection). Fish that are saved become the first generation (F1) of select brood fish. Their offspring, in turn, are referred to as the “F2 generation,” etc. The select brood fish is allowed to mate among themselves at random, and this process is then repeated in succeeding generations. Many species exhibit sexual dimorphism in that one sex grows to a larger size or grows faster. If the species does not exhibit sexual dimorphism or if selection will occur before sexual dimorphism begins, then a single cut-off value can be created for the entire population. If the species exhibits sexual dimorphism, separate cut-off values must be created for each sex, or the select population may be composed of only the larger sex.

In individual selection (also known as mass selection), all individuals are measured, and the decision to select or to cull a fish is based solely on that fish's phenotypic value. Each fish is compared to a lipid cut-off value, and fish whose phenotypic value is equal to or larger than the lipid cut-off value are saved, while fish whose phenotypic value is smaller than the lipid cut-off value are culled. A lipid cut-off value is usually based on saving a pre-determined percentage of the population. For example, the lipid content of a random sample of 100-200 fish are determined and ranked, and the value that corresponds to the desired percentile is the cut-off value. The lipid cut-off value is a pre-determined phenotypic value that can be set at top 30%, top 20%, top 10%, top 5%, top 2%, or top 1% of a population.

Family selection differs from individual selection in that the decision to save or to cull fish is conducted at the family level, and individual phenotypic values are important only as they relate to their family's mean. Two types of family selection can be applied: between-family selection and within-family selection, can be used in the methods of the invention. In between-family selection, the mean values for each family are determined, and the mean values are then ranked. Whole families are then either saved or culled. In within-family selection, each family is considered to be a temporary sub-population, and selection occurs independently within each family. When fish are measured to determine which will be saved and which will be culled, the fish in each family are ranked, and the best fish are saved from each family.

Family selection is preferably used when individual selection is inefficient because the heritability of the phenotype is small (generally h²≦0.15). When heritability is small, the heritable component of phenotypic variance is small, which means that most of the measurable differences among individuals are due to non-heritable sources of variance. By selecting at the family level, a significant portion of environmental variance can be negated, which makes it easier to identify genetic differences and to select the fish that are best because of heritable variance. The average heritabilities (h²) of lipid content in common carp is 0.14 and in channel catfish 0.23 (1983, Gjedrem, T. “Genetic variation in quantitative traits and selective breeding in fish and shellfish,” Aquaculture 33:51-72). In a preferred embodiment of the invention, the family selection method is used in selective breeding for high lipid content. Family selection is also preferably used when environmental sources of variance are uncontrollable, which can make improvement by individual selection difficult or impossible. For example, if fish cannot be spawned synchronously and if they usually spawn over a several-week to several-month period, family selection is preferred.

In another embodiment, the methods of the invention comprise a crossbreeding program involving different breeds or varieties (intraspecific crossing), or different species (interspecific crossing). Crossbreeding increases heterozygosity, and can result in heterosis (or hybrid vigor) wherein the fitness of the offspring exceeds the mean of the average values of the two parental lines. Crossbreeding can involve genetically distant parents, including those of different species or breeds, to develop a new breed with a combination of characteristics of two or more species or breeds. Crossbreeding can be used to increase the viability of a breed by introducing genetic traits for resistance to diseases or changes in environmental factors. Crossbreeding techniques that are well known, such as the techniques used in creating hybrid stripped bass, can be applied.

In a preferred embodiment of the invention, tilapia with high lipid content are produced by breeding program(s). Live tilapia are marketed in the 450 to 680 grams (1-1.5 pound) range, and yield between 30 to 39 percent whole fish to boneless fillets. Nutritive value of hybrid tilapia is considered around: 96 kcal/100 grams of raw meat, 19.2% protein and 2.3% fat by weight. Tilapia are second only to carps as the most widely farmed freshwater fish in the world. The group consists of three aquaculturally important genera: Oreochromis, Sarotherodon and Tilapia. Important commercial species include: the Mozambique or Java tilapia (Oreochromis mossambicus), blue tilapia (O. aureus), Nile tilapia (O. niloticus), Zanzibar or Wami tilapia (O. hornorum), and the redbelly tilapia (O. zilli). Many hybrid stocks constitute the bulk of the commercial production, including genetic crosses of predominantly blue tilapia (O. aureus) and ancillary O. niloticus, O. mossambicus, and O. hornorum species. Some evidence of genes from Tilapia rendalli and Sarotherodon melanotheron are also apparent. Two popular hybrids are the Florida red, a species cross between O. aureus and O. mossambicus, and the hybrid between the O. aureus and O. niloticus tilapias.

The aurea strain is principally used because of its tolerance to cold water temperatures. Hybrid tilapia are commonly sold as red or golden tilapia. The hybrids were bred for its coloration. The fish with red coloration fetch a higher price in food market because of its similarity to marine red snappers. Techniques for hybridizing Tilapia stocks are well known in the art and can be applied with high lipid content as a selection criteria to breed new tilapia stocks with greater than 2.3%, 2.5% 3%, 3.5%, 4%, 4.5%, or 5% fat by weight.

### 4.3 Genetic Engineering Methods

In another embodiment of the invention, the genetically improved fish is produced by recombinant DNA methods, wherein the DNA of an original fish is modified or foreign nucleic acid is introduced into the fish, by an exogenous recombinant DNA construct. “Nucleic acid” or “polynucleotide,” as used herein interchangeably, refers to a deoxyribonucleotide (DNA) or ribonucleotide (RNA) in either single- or double-stranded form. “Transgenic fish” refers to fish, or progeny of a fish, into which a recombinant DNA construct has been introduced, and includes fish that have developed from embryonic cells into which the construct has been introduced. Preferably, the transgenic fish of the present invention is one whose somatic and germ cells contain at least one copy of a recombinant construct of the invention. Most preferably, the recombinant construct is integrated into the fish genome. The transgenic fish or fish cell may contain a multiplicity of genomically-integrated copies of the construct. The transgenic fish of the invention is characterized by the lipid content of the fish, a part or an organ thereof, that is higher than that of a fish without the transgene, such as a wild type fish of the same species, or the parental fish that contributed the male gametes, female gametes, or zygotes, to which the transgene was introduced.

A recombinant construct is a nucleic acid molecule that is artificially introduced, or was originally artificially introduced, into an animal. The cells to which the recombinant DNA construct is introduced are referred to as “host cells.” The term artificial introduction is intended to exclude introduction of a construct through normal reproduction or genetic crosses. That is, the original introduction of a gene or trait into a line or strain of animal by breeding as described in section 4.2 is intended to be excluded.

The gene in the original fish that is to be modified is referred to as the “target gene.” The recombinant DNA construct of the invention comprises a gene, an open reading frame, and/or a gene expression control element, that play a functional role, directly or indirectly, in elevating the lipid content of the transgenic fish. The term “transgene” is used herein, to refer to the gene, open reading frame and/or gene expression regulatory region in the construct. In certain embodiments, the gene, open reading frame, and regulatory region in the construct comprise DNA sequence(s) of the target gene. In various embodiments of the invention, a regulatory region is operably linked with the gene or open reading frame to enable transcription or transcription and translation, in a fish cell. The term “operably linked” refers to a functional linkage between a promoter and a second sequence, wherein the promoter sequence initiates and mediates transcription of the DNA sequence corresponding to the second sequence. The recombinant construct can also contain DNA sequences that facilitate integration of the transgene into the genome of the fish, for example, via homologous recombination. The recombinant construct may also contain sequences that permit maintenance and replication of the construct in more than one type of host cell, such as replication origins, autonomously replicating sequences, centromere DNA, and telomere DNA. The recombinant construct may comprise selectable or screenable marker genes for isolating, identifying or tracking cells containing the transgene. Any of the cloning and expression vectors described herein may be synthesized and assembled from known DNA sequences by well known techniques in the art.

According to the invention, the target gene can be (i) a stimulator gene the expression of which is associated with an increase in lipid content; or (ii) a suppressor gene wherein its expression is associated with a decrease in lipid content. If the target gene is initially isolated from a species other than the fish species that is to be improved, the nucleic acid of an orthologous gene or a functionally homologous gene from another species can be used to make the transgene in the recombinant construct, i.e., using a functional homolog or an ortholog of a stimulator or suppressor gene from another fish, a teleost fish, a vertebrate, a mammal, or a human. Preferably, the transgene is obtained from the fish species or strain in which the recombinant construct will be introduced, or a species of fish in the same genera or family. The term “homologous” is used herein to indicate a similarity not just in nucleotide sequence but also in the function of the protein encoded by the gene, within the context of the invention. Homologous sequences are orthologous if they were separated by a speciation event: when a species diverges into two separate species, the divergent copies of a single gene in the resulting species are said to be orthologous. As used herein the term “homolog” encompasses an ortholog.

Non-limiting examples of target genes, the species of origin and their GenBank database accession numbers, are provided below. Exemplary stimulator genes include neuropeptide Y (NPY), pancreatic peptide (PP), agouti-related protein (AgRP), secretins, ghrelin, insulin, insulin-like growth factors (IGFs), orexin A, orexin B, and galanin, and their respective receptors, PPARγ, lipoprotein lipase (LPL), fat-induced transcripts 1 and 2 (FIT1, FIT2); and in particular, neuropeptide Y: AAV49168 Oreochromis sp. YC-2004 (red tilapia), AAB25269 Oncorhynchus mykiss (rainbow trout), CAB64932 Dicentrarchus labrax (European seabass), AAF71617 Ictalurus punctatus (channel catfish), AAG00549 Cyprinus carpio (common carp), AAX19943 Gadus morhua (Atlantic cod), AAX35720 Epinephelus coioides (orange-spotted grouper), AAM51821 Siniperca chuatsi (Chinese perch), ABY27301 Acipenser sinensis (Chinese sturgeon); neuropeptide Y receptors: ABS89161 Clupea harengus (Atlantic herring), and ABS89152 Acipenser baerii (Siberian sturgeon); insulin-like growth factors: CAA77264, CAA77265 Oreochromis mossambicus (Mozambique tilapia), NP_{—}001118168, Oncorhynchus mykiss (rainbow trout), ABG57072 Micropterus salmoides (largemouth bass); fish growth hormones: JE0144 Cyprinus carpio (common carp), AAL68828 Megalobrama amblycephala (Wuchang bream), CAA42022 Lates calcarifer (barramundi perch), AAT91088 fathead minnow (Pimephales promelas), AAP31126 Salvelinus alpinus (Arctic char), AAA49556 Oncorhynchus mykiss (rainbow trout); ghrelins: ABS30388 Hippoglossus hippoglossus (Atlantic halibut), BAC55160; Oreochromis mossambicus (Mozambique tilapia), ABN13418 Oreochromis urolepis hornorum (Wami tilapia), BAC65151 Oreochromis niloticus (Nile tilapia), BAB96565 Anguilla japonica (Japanese eel), ACD13783 Salmo salar (Atlantic salmon), AAV65509 Acanthopagrus schlegelii (black porgy), AAN16216 Carassius auratus (goldfish), BAF95542 Cyprinus carpio (common carp), NP_{—}001118060 Oncorhynchus mykiss (rainbow trout), ABG49130 Dicentrarchus labrax (European seabass); PPARγ: CAB51396 Platichthys flesus (European flounder), AAT85618 Sparus aurata (gilthead seabream); lipoprotein lipases: AAK69707 Oncorhynchus mykiss (rainbow trout), ACG63500 Pelteobagrus vachellii, CAL69901 Dicentrarchus labrax (European seabass), AAH64296 Danio rerio (zebrafish), AF98179 Thunnus orientalis (Pacific bluefin tuna); FIT1: NP_{—}001013343 Danio rerio (zebrafish); FIT2: NP_{—}001018334 Danio rerio (zebrafish); Melanocortin receptor 4: NP_{—}775385 Danio rerio (zebrafish), NP_{—}001027732 Takifugu rubripes; agouti-related protein: CAD88211 Carassius auratus (goldfish); orexins: ABF29871 Gadus morhua (Atlantic cod), ABH04375 Danio rerio; orexin receptors: AB061386 Danio rerio, ABQ40389 Thalassoma pavo; galanins: AA065775, AA065776, AA065778, AA065779 Carassius auratus, P47213 Oncorhynchus mykiss (rainbow trout), AAB32703 Amia calva (bowfin); insulins and IGFs: 544470 Polyodon spathula (Mississippi paddlefish), P04667 Oncorhynchus keta (chum salmon), P68991 Chimaera monstrosa (rabbit fish), P01339 Thunnus thynnus (northern bluefin tuna), CAA77265 Oreochromis mossambicus (Mozambique tilapia), NP_{—}571900 Danio rerio, NP_{—}001118169 Oncorhynchus mykiss.

Exemplary suppressor genes include leptin, cholecystokinin (CCK), cocaine and amphetamine-regulated transcript (CART), corticotropin-releasing factor (CRF), bombesin (or gastrin-releasing peptide), alpha-melanocyte-stimulating hormone (alpha-MSH), tachykinins, glucagon-like peptide-1 (GLP-1), urotensin I, and somatostatin, and their respective receptors, PPARα, PPARδ, β-glucocerebrosidase, α-galactosidase, β-N-acetylhexosaminidase A, acid sphingomyelinases, NPC1 and NPC2; and in particular, leptins: ACF23048 Ctenopharyngodon idella (Chinese grass carp), AB193548 Oryzias latipes, AAZ66785 Ictalurus punctatus (channel catfish); leptin receptors: BAG09232 Oncorhynchus mykiss (rainbow trout), CAJ33891 Danio rerio (zebrafish), ABC86922 Oryzias melastigma (Indian medaka), ACG69477 Carassius auratus (goldfish); somatostatins: AAU93565 Epinephelus coioides (orange-spotted grouper), AAI62710 Danio rerio (zebrafish); PPARα CAI54224, CAI54225 Dicentrarchus labrax (European seabass), AAT85613 Sparus aurata (gilthead seabream), CAJ76701 Salmo salar (Atlantic salmon); PPARδ: AAT85615 Sparus aurata (gilthead seabream), AAK76392 Danio rerio (zebrafish); β-glucocerebrosidase: ACI69345 Salmo salar (Atlantic salmon); α-galactosidase: CAC44626 Takifugu rubripes; β-N-acetylhexosaminidase: ACI66373, ACI33266 Salmo salar (Atlantic salmon); acid sphingomyelinase: NP_{—}035551 Mus musculus; Proopiomelanocortin: NP_{—}852103 Danio rerio (zebrafish); CCK: BAE16613 Seriola quinqueradiata (Japanese amberjack).

Several non-limiting examples of target genes are described below. The description should in no way be construed, however, as limiting the broader scope of the invention. To illustrate the functional conservation of a suppressor gene, the cDNA encoding a homolog of mammalian leptin has been isolated from the liver of pufferfish, and homologs have also been identified in the sequence databases of salmon, medaka, and Tetraodon (2005, Kurokawa et al., Peptides, 5:745-750). Administration of leptin to obese animals produced weight loss by decreasing appetite and increasing the rate of fat metabolism. Similar results had been obtained from using recombinant rainbow trout leptin (2008, Marashita et al., Comp. Biochem. Physiol. B, Biochem. Mol. Biol. 150 (4), 377-384) indicating that the neuroendocrine pathways that control feeding is highly conserved among vertebrates. Accordingly, the vertebrate leptin genes and its fish homologs, are useful as suppressor genes of the invention.

Another example of the functional conservation of mammalian and fish genes also illustrates the usefulness of a zebrafish model. It is known that reduction of melanocortin 4 receptor (MC4-R) signaling, caused by mutations in either the POMC or MC4-receptor genes or by overexpression of MC4-receptor antagonists like agouti or agouti-related protein (AgRP), causes obesity in mammals. Transgenic zebrafish overexpressing the endogenous melanocortin antagonist AgRP also exhibit obesity, increased linear growth, and adipocyte hypertrophy (2007, Song and Cone, FASEB Journal. 21:2042-2049). While the reported zebrafish system is used for genetic analysis of energy homeostasis, the invention provides the overexpression of AgRP and other stimulator genes for creating obese fish that can be used to harvest algae. Unlike drug discovery programs that use zebrafish to find drugs to reduce obesity in humans, the objective of the invention is to identify factors and culture conditions that encourage obesity in fish, preferably in another fish species. Therefore, the invention encompasses a transgenic zebrafish comprising an overexpressing transgene that is a stimulator gene. The invention also encompasses using the AgRP gene as a transgene in transgenic fishes but the transgenic fish is not a zebrafish.

The ability to store lipid in the form of cytoplasmic triglyceride droplets is apparently conserved from yeast to human. The expression of a family of lipogenesis genes FIT1 and FIT2 (fat-inducing transcripts) was studied in mouse cells and zebrafish (2008, Kadereit et al., Proc Natl Acad Sci USA. 105(1):94-9). Short hairpin RNA silencing of FIT2 in mouse 3T3-LI adipocytes prevents accumulation of lipid droplets. Depletion of FIT2 in zebrafish by the use of morpholino antisense oligonucleotide blocks diet-induced accumulation of lipid droplets in the larval intestine and liver. The results indicate that the FIT family of genes are stimulator genes and can be used as a transgene in the transgenic fishes of the invention. Accordingly, the invention encompasses a transgenic fish comprising an overexpressing transgene that is a member of the FIT gene family.

The nucleic acids described in the sequence database records of target genes can be used to construct the transgene or to isolate a homolog from another species. Homologs of such sequences in other species can be identified and readily isolated, without undue experimentation, by bioinformatics and molecular biological techniques well known in the art. The sequences and their identifiers can be used to retrieve the sequences of homologs in sequence databases. A variety of such databases are available to those skilled in the art, including GenBank and GenSeq. In various embodiments, the databases are screened to identify nucleic acids with at least 95%, at least 90%, at least 85%, at least 80%, at least 70%, at least 60%, at least 50%, or at least 40% nucleotide sequence identity to a target gene sequence, or a portion thereof. In other embodiments, the databases are screened to identify polypeptides having at least 99%, at least 95%, at least 90%, at least 85%, at least 80%, at least 70%, at least 60%, at least 50%, at least 40%, at least 30%, or at least 25% amino acid identity or similarity to a polypeptide encoded by the target genes of the invention.

Homologous genes of the invention share a certain degree of sequence identity at the amino acid level or nucleic acid level. The degree of identity is preferably determined on the amino acid sequence of a mature polypeptide, i.e. without taking any leader sequence into consideration. The percentage of sequence identity between two sequences is determined by comparing two optimally aligned sequences over a window of comparison of at least 20 positions. Optimal alignment of sequences for comparison may be conducted by the local homology algorithm of Smith and Waterman, Adv. Appl. Math., 2:482 (1981), by the homology alignment algorithm of Needleman and Wunsch, J. Mol. Biol., 48:443 (1970), by the search for similarity method of Pearson and Lipman, Proc. Natl. Acad. Sci. (U.S.A.), 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

Techniques for isolating homologous gene sequences from another species by hybridization and/or polymerase chain reaction are well known in the art. Substantial nucleic acid sequence identity exists when a nucleic acid segment will hybridize, under selective hybridization conditions, to a complement of another nucleic acid strand. Selectivity of hybridization exists when hybridization occurs that is more selective than a total lack of specificity. To clone a stimulator or suppressor gene homolog, a labeled nucleic acid probe (based on known sequence) can be used to screen a cDNA library constructed from mRNA obtained from appropriate fish cells or tissues (e.g., liver, muscle, ovary, testes, brain) derived from the fish of interest. Low, moderate and high stringency conditions are well known to those of skill in the art, and will vary predictably depending on the base composition of the particular nucleic acid sequence and on the specific organism from which the nucleic acid sequence is derived. For cross species hybridization, low stringency conditions are preferred. For hybridization of DNA from species within the same family or genus, moderately stringent conditions are preferred. For guidance regarding such conditions see, for example, Maniatis et al., 1990, Molecular Cloning, A Laboratory Manual, Second Edition, Cold Spring Harbor Press, N.Y., pp. 9.47-9.57; and Ausubel et al., 1989, Current Protocols in Molecular Biology, Green Publishing Associates and Wiley Interscience, N.Y. Various other stringency conditions which promote DNA hybridization can be used. For example, hybridization in 6×SSC at about 45° C., followed by washing in 2×SSC at 50° C. may be used. Alternatively, the salt concentration in the wash step can range from low stringency of about 5×SSC at 50° C., to moderate stringency of about 2×SSC at 50° C., to high stringency of about 0.2×SSC at 50° C. In addition, the temperature of the wash step can be increased from low stringency conditions at room temperature, to moderately stringent conditions at about 42° C., to high stringency conditions at about 65° C. Other conditions include, but are not limited to, hybridizing at 68° C. in 0.5M NaHPO₄ (pH7.2)/1 mM EDTA/7% SDS, or hybridization in 50% formamide/0.25M NaHPO₄ (pH 7.2)/0.25 M NaCl/1 mM EDTA/7% SDS; followed by washing in 40 mM NaHPO₄ (pH 7.2)/1 mM EDTA/5% SDS at 50° C. or in 40 mM NaHPO₄ (pH7.2) 1 mM EDTA/1% SDS at 50° C. Both temperature and salt may be varied, or alternatively, one or the other variable may remain constant while the other is changed.

Polymerase chain reaction (PCR) can also be used to isolate by amplification the DNA of a homolog from genomic DNA or cDNA of the species of interest. This approach is particularly useful when only homologous sequence information is available. The cDNA can be obtained by reverse transcription of mRNA prepared from the tissue in which the gene is expressed. Oligonucleotide primers representing known sequences, preferably representing at least part of the conserved segments of strong homology between the desired genes of different species, can be used. Several different degenerate primers can be used under different stringency of hybridization conditions to prime the PCR reactions, to allow for greater or lesser degrees of nucleotide sequence similarity between the known nucleotide sequence and the nucleic acid homolog being isolated. After successful amplification of a segment of a homolog, that segment may be molecularly cloned and sequenced, and utilized as a probe to isolate a complete cDNA or genomic clone. This, in turn, will permit the assembly of the recombinant transgene. In this fashion, homologous stimulator or suppressor genes may be isolated.

Standard recombinant DNA techniques, such as restriction digestion and ligation, are used to assemble the construct comprising the transgene. Methods described in detail infra are for illustration only and not by way of limitation. Depending on the strategy, many designs of the construct may be adopted, including but not limited to, plasmids, modified viruses, or artificial chromosomes. Various cloning vectors and expression systems that are commercially available may also be used according to the manufacturer's instructions. To facilitate expression of a transgene in the transgenic fish, the recombinant construct comprises the transgene operably associated with gene expression regulatory regions that are functional in the fish's cells. Such regions comprise promoters and optionally enhancers and/or transcriptional terminators. Constitutive or inducible regulatory regions may be used for expression of the transgene. It may be desirable to use inducible promoters to control the high level expression of the transgene once the expression construct is introduced into fish cells in vivo. It may also be desirable to use promoters that are not tissue specific, thus allowing ectopic expression of the transgene in the fish. If the activity of the transgene is desired in a specific tissue or organ of the fish (e.g., liver, testes, ovary, muscle), tissue-specific or organ-specific regulatory regions may be used. The regulatory regions can be of a variety of origins, e.g., native to the host species, derived from a homolog in another species, or synthetic.

In one embodiment, the expression of a stimulator gene is increased in the transgenic fish. The expression of stimulator gene can be increased by increasing the copy number of the native stimulator gene, or introducing into the animal a homolog of the stimulator gene. This can be accomplished by inserting one or more copies of the native stimulator gene or a homolog thereof with the appropriate gene expression regulatory regions (as the transgene) into the recombinant construct, and introducing the construct into the fish, such that the extra copy of stimulator gene or the homolog of the stimulator gene is expressed in the fish. The transgene thus comprises an expressible stimulator gene. The regulatory region of the native stimulator gene can be used. Alternatively, a regulatory region of another host or non-host gene that has a similar or greater activity than the native stimulator gene and/or a different range of tissue specificity can be used. In another approach, the expression of stimulator gene can be increased by deregulating expression of the native stimulator gene. This can be accomplished by replacing the regulatory region of the native gene with that of another host or non-host gene that has a greater activity and/or that has activity in a broader range of tissues. Ectopic and/or constitutive expression of a native stimulator gene or a homolog thereof in the transgenic fish is contemplated.

In another embodiment, the invention provides a transgenic fish wherein the expression of a suppressor gene is decreased, thereby increasing the lipid content of the fish. The decrease of suppressor gene expression can be accomplished by knocking out the suppressor gene in the fish genome, or use of antisense polynucleotides, including RNA interference, to knockdown suppressor gene expression. In one embodiment, a transgenic fish is produced by promoting homologous recombination between a target suppressor gene including the regulatory regions in its chromosome and an exogenous transgene that has been rendered biologically inactive (preferably by insertion of a heterologous sequence, preferably, a selectable marker, in the coding region).

Another approach is the use of heritable dsRNA-producing constructs to achieve RNA interference (RNAi) in fish. RNAi refers to interference with or destruction of the product of a target gene by introducing a double stranded RNA (dsRNA) that is homologous to the product of a target gene. This may be accomplished using any of the techniques reported in the art, for instance by transfecting a nucleic acid construct encoding a stem-loop or hairpin RNA structure into the genome of the fish, or by expressing a transfected nucleic acid construct having homology for a target gene from between convergent promoters, or as a head to head or tail to tail duplication from behind a single promoter. Any similar construct may be used so long as it produces a single RNA having the ability to fold back on itself and produce a dsRNA (e.g., short hairpin RNA or shRNA), or so long as it produces two separate RNA transcripts which then anneal to form a dsRNA having homology to a target gene. Absolute homology is not required for RNAi, with a lower threshold being described at about 85% homology for a dsRNA of about 100-200 base pairs, and for longer dsRNAs, i.e., 300 to 1000 base pairs, having at least about 75% homology to the target gene. RNA-encoding constructs that express a single RNA transcript designed to anneal to a separately expressed RNA, or single constructs expressing separate transcripts from convergent promoters, are preferably at least about 100 nucleotides in length. RNA-encoding constructs that express a single RNA designed to form a dsRNA via internal folding are preferably at least about 200 nucleotides in length. The promoter used to express the dsRNA-forming construct may be any type of promoter if the resulting dsRNA is specific for a gene product in the cell lineage targeted for interference. Alternatively, the promoter may be lineage specific in that it is only expressed in cells of a particular development lineage.

The transgenic fish of the invention are produced by introducing a recombinant construct of the invention into cells of a fish, preferably embryonic cells, and most preferably in a single cell embryo. Where the transgene construct is introduced into embryonic cells, the transgenic fish is obtained by allowing the embryo to develop into a fish. Introduction of constructs into embryonic cells of fish, and subsequent development of the fish, are simplified by the fact that embryos develop outside of the parent fish. A recombinant construct can be introduced into embryonic fish cells using any suitable technique. Many techniques for such introduction of exogenous genetic material have been demonstrated in fish and other animals. These include microinjection (described by, for example, Gulp et al. (1991) Proc Natl Acad Sci USA 88, 7953-7957), electroporation (described by, for example, Inoue et al. (1990), Cell. Differ. Develop. 29, 123-128; Muller et al. (1993), FEES Lett. 324, 27-32; Murakami et al. (1994), Biotechnol 34, 35-42; Muller et al. (1992), Mol. Mar. Biol. Biotechnol. 1, 276-281; and Symonds et al. (1994), Aquaculture 119, 313-327), particle gun bombardment (Zelenin et al. (1991), FEES Lett. 287, 118-120), retroviral vectors (Lu et al (1997). Mol Mar Biol Biotechnol 6, 289-95), and the use of liposomes (Szelei et al. (1994), Transgenic Res. 3,116-119).

Fish embryos or embryonic cells can generally be obtained by collecting eggs immediately after they are laid. It is generally preferred that the eggs be fertilized prior to or at the time of collection. This is preferably accomplished by placing a male and female fish together in a tank that allows egg collection under conditions that stimulate mating. After collecting eggs, it is preferred that the embryo be exposed for introduction of genetic material by removing the chorion. This can be done manually or, preferably, by using a protease such as pronase. A fertilized egg cell prior to the first cell division is considered a one-cell embryo, and the fertilized egg cell is thus considered an embryonic cell. After introduction of the transgene construct, the embryo is allowed to develop into a fish. This generally need involve no more than incubating the embryos under the same conditions used for incubation of eggs. However, the embryonic cells can also be incubated briefly in an isotonic buffer. If appropriate, expression of an introduced transgene construct can be observed during development of the embryo. Fish harboring a transgene can be identified by any suitable means. For example, the genome of potential transgenic fish can be probed for the presence of construct sequences. To identify transgenic fish actually expressing the transgene, the presence of an expression product can be assayed. Several techniques for such identification are known and used for transgenic animals and most can be applied to transgenic fish. Probing of potential or actual transgenic fish for nucleic acid sequences present in or characteristic of a transgene construct is preferably accomplished by Southern blotting or Northern blotting. Also preferred is detection using polymerase chain reaction (PCR) or other sequence-specific nucleic acid amplification techniques.

A transgenic fish of the invention can be hemizygous for the transgene, which is the preferred state for maintenance of fish lines. Alternatively, hemizygous fish can be crossed with each other to produce homozygous fish or fish lines. Homozygous diploids can also be produced by other methods, e.g., interruption of the second meiotic divisions with hydrostatic pressure using a French press. The disclosed recombinant constructs are preferably integrated into the genome of the fish. However, the disclosed transgene construct can also be constructed as an artificial chromosome. In another embodiment, the invention includes a genetically identical population of transgenic fish, each of whose somatic and germ cells contain at least one genetically integrated copy of a recombinant construct of the invention. The genetically identical population is a unisex population and can be male or female.

The invention further provides a transgenic fish gamete, including an transgenic fish egg or sperm cell, a transgenic fish embryo, and any other type of transgenic fish cell or cluster of cells, whether haploid, diploid, triploid or other zygosity having at least one genomically integrated copy of a recombinant construct of the invention. The invention further includes a cell line derived from a transgenic fish embryo or other transgenic fish cell of the invention, which contains at least one copy of a recombinant construct of the invention. Progeny of a transgenic fish containing at least one genomically integrated copy of the construct, and transgenic fish derived from a transgenic fish egg, sperm, embryo or other fish cell of the invention, are also included in the invention. In various embodiments, a transgenic embryo of the invention can develop into a transgenic fish of the invention; a transgenic egg of the invention can be fertilized to create a transgenic embryo of the invention that develops into a transgenic fish of the invention; a transgenic sperm cell of the invention can be used to fertilize an egg to create a transgenic embryo of the invention that develops into a transgenic fish of the invention; and a transgenic cell of the invention can be used to clone a transgenic fish of the invention. In some embodiments of the invention, the transgenic fish is sterile.

### 4.4 Selection Methods

The selection methods of the present invention are used to identify fish that feed on algae and result in a high lipid content. The methods do not distinguish whether the lipids are synthesized by the fish from carbohydrates or protein, or obtained directly from the algae. Any method for extracting lipids from fish tissue, and any method for quantitation of the extracted lipids known in the art can be applied. A commonly used technique is described in Bligh and Dyer (1959, “A rapid method of total lipid extraction and purification,” Canadian J Biochem. Physiol. 37:911-917). This technique involves homogenizing wet fish tissue with a mixture of chloroform and methanol in proportions that a miscible system is formed with the water in the tissue, diluting the homogenate with chloroform and water to form two layers, wherein the chloroform layer contains lipids and the methanol layer contains non-lipids.

The invention contemplates elevation of the lipid content in the entire fish, or only in certain part(s) or organ(s) of a fish, such as but not limited to, fish fillet, fish viscera, muscle, head, liver, guts, bones, testes, and ovary. In certain embodiments, a change in the relative abundance of different lipids and/or the appearance of new lipids are also expected. Accordingly, in certain embodiments, not the entire fish but only a part or an organ of the fish is used in determination of lipid content and lipid quality. The fish that are to be selected can be a population that emerges from a breeding program described in section 4.4 or a genetic engineering effort described in section 4.5.

Generally, the methods comprise providing an algal composition to a population of fish that are to be selected, allowing the fish to feed on the algae, and measuring the lipid content of the fish after a period of time Fish that meet or exceed a cut-off value are saved. As described in section 4.5, the algal composition can be a mixture of different species of algae. In one embodiment, the algal composition comprises more than one type or one taxonomic group (such as a genus or a species) of algae, wherein the types or groups of algae, and the proportions of each type or groups is defined. Such an algal composition can be made by mixing two or more monocultures or cultures with a dominant major species to arrive at the defined proportions. The use of a defined algal composition can reduce variability in nutrients when conducting selection. After an improved fish has been identified using an algal composition, the invention provides that the types/groups of algae or the proportions of each types/groups be adjusted to maximize the gain in lipid content in the improved fish. It is contemplated that the algal composition can be designed and optimized to enhance the growth of and/or accumulation of lipids in a breed of improved fish of the invention.

The selection methods also comprise making an algae composition accessible to the fish in a controlled manner, preferably under conditions similar to algae harvesting. Any method by which the algae and fish of the invention are brought into proximity of each other, such that the fishes can ingest the algae, can be used. The algae and the fish can be kept separately for at least a period of time before the algae are fed to the fish. The concentration of an algal composition can range from about 0.05 g/L, about 0.1 g/L, about 0.2 g/L, about 0.5 g/L to about 1.0 g/L. An alternative system to assess algal concentration that measures chlorophyll-a concentration (μg/L) can be used similarly. Generally, the fishes are selected to maintain a low FCR, which can increase the net energy produced by the system. Thus, controlling the concentration of algae on which the fishes feed can be useful for optimizing the FCR, such as by reducing the FCR in a system. In some embodiments, the fish has an FCR of less than about 3, less than about 2, less than about 1.5, less than about 1.0, less than about 0.8, or less than 0.6. FCR is one of the quantitative phenotypes that can be selected in the methods of the invention, given a defined diet of algae, feeding regimen, and harvest conditions.

Depending on the growth rate and life cycle of the fish under selection, they can be gathered at any time after they have fed on the algae and gained sufficient biomass for fish oil and fishmeal processing. The fish under selection can be fed with the algae and kept for about 7 to 14 days, about 10 to 30 days, about 30 to 90 days, about 12 to 24 weeks, or about 6 to 24 months. The fish can be gathered for measurements by any methods or means known in the art.

A cut-off value, measurable in lipid content, is determined after a period of time in culture. For example, the cut-off value can be the 2-week weight, 2-week length, 2-week moisture content, 2-week fat content, 4-week weight, 4-week length, 4-week moisture content, 4-week fat content, 8-week weight, 8-week length, 8-week moisture content, 8-week fat content, 3-month weight, 3-month length, 3-month moisture content, 3-month fat content, 6-month weight, 6-month length, 6-month moisture content, 6-month fat content, 12-month weight, 12-month length, 12-month moisture content, or 12-month fat content. It is contemplated that, depending on the species of fish, the weight, body length, body depth, moisture content, ash content, and other parameters known in the art, can be used as a surrogate indicator of lipid content. The correlation of lipid content and such a indicator can be determined for a particular fish breed by routine experimentation. The use of a surrogate indicator can save the fish from being sacrificed, reduce harm to the fish, or reduce the cost of doing a large scale selection. See Table 1 which shows the lipid content and moisture content for a number of freshwater fish species found in North America.

| | Moisture (%) | Lipid (%) |
|---|---|---|
| Species | Average | Average |
| Alewife | 72.8 | 10.2 |
| Largemouth Bass | 79.1 | 1.3 |
| Smallmouth Bass | 77.6 | 2.3 |
| White Bass | 79.6 | 4.2 |
| Bowfin | 78.0 | 2.7 |
| Bigmouth Buffalo | 74.4 | 5.5 |
| Smallmouth Buffalo | 75.4 | 6.0 |
| Bluntnose Minnow | 69.5 | 9.2 |
| Common Carp | 74.5 | 6.3 |
| Carp | 74.3 | 6.8 |
| River Carpsucker | 77.5 | 6.1 |
| Channel Catfish | 77.0 | 5.8 |
| Flathead Catfish | 80.7 | 2.1 |
| Freshwater Drum | 75.3 | 4.8 |
| Gizzard Shad | 68.9 | 7.2 |
| Goldfish | 73.9 | 4.9 |
| Northern Hog Sucker | 76.9 | 3.3 |
| Quillback | 77.5 | 3.8 |
| Shorthead Redhorse | 76.2 | 5.3 |
| Golden Redhorse | 78.9 | 4.2 |
| Blacktail Redhorse | 77.7 | 3.2 |
| Paddlefish | 78.2 | 4.0 |
| Chinook Salmon | 75.2 | 3.1 |
| Coho Salmon | 75.0 | 3.3 |
| Golden Shiner | 71.1 | 5.2 |
| Spotted Sucker | 78.6 | 3.0 |
| White Sucker | 78.9 | 3.0 |
| Longear Sunfish | 73.7 | 2.7 |
| Brown Trout | 68.7 | 10.7 |
| Lake Trout | 62.2 | 19.6 |
| Rainbow (Steelhead) Trout | 69.7 | 7.9 |
| Walleye | 79.1 | 1.1 |
| Yellow Perch | 75.8 | 2.1 |

When the proportions of freshwater fish body lipids and moisture are summed up and averaged, the number tends to be about 80% of fish body mass. This relation holds true across a complete roster of fish body parts, organs, tissues and species. Accordingly, there is a negative correlation between the percentages of body lipid and body moisture in fish. Moreover, the relationship appears to hold even under varying conditions of feeding, growth and gonad development. Because it is simpler to measure moisture than either protein or lipid content, moisture content is the most commonly used independent variable in predictive equations.

### 4.5. Algae

The algae described below are algae that can be harvested by fish cultured by the methods of the invention, or by the genetically improved fish of the invention. The algae can also be used in selection methods of the invention to identify an improved fish.

As used herein the term “algae” refers to any organisms with chlorophyll and a thallus not differentiated into roots, stems and leaves, and encompasses prokaryotic and eukaryotic organisms that are photoautotrophic or photoauxotrophic. The term “algae” includes macroalgae (commonly known as seaweed) and microalgae. For certain embodiments of the invention, algae that are not macroalgae are preferred. The terms “microalgae” and “phytoplankton,” used interchangeably herein, refer to any microscopic algae, photoautotrophic or photoauxotrophic eukaryotes (such as, protozoa), photoautotrophic or photoauxotrophic prokaryotes, and cyanobacteria (commonly referred to as blue-green algae and formerly classified as Cyanophyceae). The use of the term “algal” also relates to microalgae and thus encompasses the meaning of “microalgal.” The term “algal composition” refers to any composition that comprises algae, such as an aquatic composition, and is not limited to the body of water or the culture in which the algae are cultivated. An algal composition can be an algal culture, a concentrated algal culture, or a dewatered mass of algae, and can be in a liquid, semi-solid, or solid form. A non-liquid algal composition can be described in terms of moisture level or percentage weight of the solids. An “algal culture” is an algal composition that comprises live algae. The microalgae used in the invention are also encompassed by the term “plankton” which includes phytoplankton, zooplankton and bacterioplankton. For certain embodiments of the invention, an algal composition or a body of water comprising algae that is substantially depleted of zooplankton is preferred since many zooplankton consume phytoplankton. However, it is contemplated that many aspects of the invention can be practiced with a planktonic composition, without isolation of the phytoplankton, or removal of the zooplankton or other non-algal planktonic organisms. The methods of the invention can be used with a composition comprising plankton, or a body of water comprising plankton.

Algae inhabit all types of aquatic environment, including but not limited to freshwater (less than about 0.5 parts per thousand (ppt) salts), brackish (about 0.5 to about 31 ppt salts), marine (about 31 to about 38 ppt salts), and briny (greater than about 38 ppt salts) environment. As the present invention can be practiced in any of such aquatic environments, freshwater species, marine species, and/or species that thrive in varying and/or intermediate salinities or nutrient levels, can be used. The algae used in the algal culture can be obtained initially from environmental samples of natural or man-made environments, and may contain a mixture of prokaryotic and eukaryotic organisms, wherein some of the minor species may be unidentified. Freshwater filtrates from rivers, lakes; seawater filtrates from coastal areas, oceans; water in hot springs or thermal vents; and lake, marine, or estuarine sediments, can be used to source the algae. The samples may also be collected from local or remote bodies of water, including surface as well as subterranean water. Endemic or indigenous species are generally preferred over introduced species where an open farming system is used. Endemic or indigenous species may be enriched or isolated from water samples obtained locally (relative to the site of the culture system). It can also be beneficial to deploy algae and fishes from a local aquatic trophic system in an open farming system. Depending on the location of the algae culture system, algae obtained from tropical, subtropical, temperate, polar or other climatic regions can be used. In certain open farming systems, the algae in an algal composition may not all be cultivable under laboratory conditions, and not all the algae in an algal composition have to be fully characterized in order to be utilized in the present invention.

According to the invention, one or more species of algae will be present in the algal culture or algal composition that is to be harvested by fish. In one embodiment of the invention, the algal culture is a monoculture, wherein only one species of algae is grown. However, in many open farming systems, it may be difficult to avoid the presence of other algae in the water. Accordingly, a monoculture may comprise about 0.1% to 2% of algae species other than the intended species, i.e., up to 98% to 99.9% of the algal cells in a monoculture are of one species. In another embodiments, the algal culture is a mixed culture that comprises one or several dominant species of algae. Microalgal species can be identified by microscopy and enumerated by counting visually or optically, or by techniques such as but not limited to microfluidics and flow cytometry, which are well known in the art. A dominant species is one that ranks high in the number of algal cells, e.g., the top one to five species with the highest number of cells relative to other species. The one or several dominant algae species may constitute greater than about 10%, about 20%, about 30%, about 40%, about 50%, about 60%, about 70%, about 80%, about 90%, about 95%, about 97%, about 98% of the algae present in the culture. In certain embodiments, several dominant algae species may each independently constitute greater than about 10%, about 20%, about 30%, about 40%, about 50%, about 60%, about 70%, about 80% or about 90% of the algae present in the culture. Many other minor species of algae may also be present in such culture but they may constitute in aggregate less than about 50%, about 40%, about 30%, about 20%, about 10%, or about 5% of the algae present. In various embodiments, one, two, three, four, or five dominant species of algae are present in a culture. Accordingly, a mixed algal culture or an algae composition can be described and distinguished from other cultures or compositions by the dominant species of algae present. The composition and culture can be further described by the percentages of cells that are of dominant species relative to minor species, or the percentages of each of the dominant species. It is to be understood that mixed algal cultures or compositions having the same genus or species of algae may be different by virtue of the relative abundance of the various genus and/or species present.

It is contemplated that many different algal cultures can be harvested efficiently by the methods of the invention. In specific embodiments, algae of a particular taxonomic group, genera or species, may be less preferred. Such algae, including one or more that are listed below, may be specifically excluded as a dominant species in a culture. However, it should also be understood that in certain embodiments, such algae may be present as a contaminant especially in an open farming system, or as a non-dominant group or minor species. Such algae may be present in negligent numbers, or substantially diluted given the volume of the culture. The presence of such algal genus or species in a culture is distinguishable from cultures where such genus or species are dominant, or constitute the bulk of the algae.

In certain embodiments, an algal composition comprising a combination of different groups of algae is used in the invention. The algal composition can be prepared by mixing a plurality of different algal cultures. The different groups of algae can be present in defined proportions. The combination and proportion of different algae in the algal composition can be designed to enhance the growth and/or accumulation of lipids of the improved fish. A microalgal composition of the invention can comprise predominantly microalgae of a selected size range, such as but not limited to, below 2000 μm, about 200 to 2000 μm, above 200 μm, below 200 μm, about 20 to 2000 μm, about 20 to 200 μm, above 20 μm, below 20 μm, about 2 to 20 μm, about 2 to 200 μm, about 2 to 2000 μm, below 2 μm, about 0.2 to 20 μm, about 0.2 to 2 μm or below 0.2 μm

In various embodiments, one or more species of algae belonging to the following phyla can be harvested by the methods of the invention: Cyanobacteria, Cyanophyta, Prochlorophyta, Rhodophyta, Glaucophyta, Chlorophyta, Dinophyta, Cryptophyta, Chrysophyta, Prymnesiophyta (Haptophyta), Bacillariophyta, Xanthophyta, Eustigmatophyta, Rhaphidophyta, and Phaeophyta. In certain embodiments, algae in multicellular or filamentous forms, such as seaweeds or macroalgae, many of which belong to the phyla Phaeophyta or Rhodophyta, are less preferred. In many embodiments, algae that are microscopic, are preferred. Many such microalgae occurs in unicellular or colonial form.

In certain embodiments, the algal composition to be harvested by the methods of the invention comprises cyanobacteria (also known as blue-green algae) from one or more of the following taxonomic groups: Chroococcales, Nostocales, Oscillatoriales, Pseudanabaenales, Synechococcales, and Synechococcophycideae. Non-limiting examples include Gleocapsa, Pseudoanabaena, Oscillatoria, Microcystis, Synechococcus and Arthrospira species.

In certain embodiments, the algal composition comprises algae from one or more of the following taxonomic classes: Euglenophyceae, Dinophyceae, and Ebriophyceae. Non-limiting examples include Euglena species and the freshwater or marine dinoflagellates.

In certain embodiments, the algal composition comprises green algae from one or more of the following taxonomic classes: Micromonadophyceae, Charophyceae, Ulvophyceae and Chlorophyceae. Non-limiting examples include species of Borodinella, Chlorella (e.g., C. ellipsoidea), Chlamydomonas, Dunaliella (e.g., D. salina, D. bardawil), Franceia, Haematococcus, Oocystis (e.g., O. parva, O. pustilla), Scenedesmus, Stichococcus, Ankistrodesmus (e.g., A. falcatus), Chlorococcum, Monoraphidium, Nannochloris and Botryococcus (e.g., B. braunii). In certain embodiments, Chlamydomonas reinhardtii are less preferred.

In certain embodiments, the algal composition comprises golden-brown algae from one or more of the following taxonomic classes: Chrysophyceae and Synurophyceae. Non-limiting examples include Boekelovia species (e.g. B. hooglandii) and Ochromonas species.

In certain embodiments, the algal composition comprises freshwater, brackish, or marine diatoms from one or more of the following taxonomic classes: Bacillariophyceae, Coscinodiscophyceae, and Fragilariophyceae. Preferably, the diatoms are photoautotrophic or auxotrophic. Non-limiting examples include Achnanthes (e.g., A. orientalis), Amphora (e.g., A. coffeiformis strains, A. delicatissima), Amphiprora (e.g., A. hyaline), Amphipleura, Chaetoceros (e.g., C. muelleri, C. gracilis), Caloneis, Camphylodiscus, Cyclotella (e.g., C. cryptica, C. meneghiniana), Cricosphaera, Cymbella, Diploneis, Entomoneis, Fragilaria, Hantschia, Gyrosigma, Melosira, Navicula (e.g., N. acceptata, N. biskanterae, N. pseudotenelloides, N. saprophila), Nitzschia (e.g., N. dissipata, N. communis, N. inconspicua, N. pusilla strains, N. microcephala, N. intermedia, N. hantzschiana, N. alexandrina, N. quadrangula), Phaeodactylum (e.g., P. tricornutum), Pleurosigma, Pleurochrysis (e.g., P. carterae, P. dentata), Selenastrum, Surirella and Thalassiosira (e.g., T. weissflogii).

In certain embodiments, the algal composition comprises planktons that are characteristically small with a diameter in the range of 1 to 10 μm, or 2 to 4 μm. Many of such algae are members of Eustigmatophyta, such as but not limited to Nannochloropsis species (e.g. N. salina).

In certain embodiments, the algal composition comprises one or more algae from the following groups: Coelastrum, Chlorosarcina, Micractinium, Porphyridium, Nostoc, Closterium, Elakatothrix, Cyanosarcina, Trachelamonas, Kirchneriella, Carteria, Crytomonas, Chlamydamonas, Planktothrix, Anabaena, Hymenomonas, Isochrysis, Pavlova, Monodus, Monallanthus, Platymonas, Pyramimonas, Stephanodiscus, Chroococcus, Staurastrum, Netrium, and Tetraselmis.

In certain embodiments, any of the above-mentioned genus and species of algae may independently be less preferred as a dominant species in, or excluded from, an algal composition of the invention.

### 4.6 Culture Methods

The present invention also encompasses culturing methods that can be used to boost the level of lipids in fish. As lipids are the primary energy reserve for many fishes, it is accumulated under certain environments and during certain life stages.

In one embodiment, the culture methods seek to emulate growing environments under which the fish accumulate lipids, such as but not limited to, lower water temperature as experienced by the fish in temperate regions during cold seasons.

In another embodiment, the culture methods comprise administering to the fish a biological agent, such as a hormone, to control its sexual differentiation or sexual maturation. The hormones that govern the sexual maturation process include but is not limited to lutenizing hormone (LH), follicle-stimulating hormone (FSH), and gonadotropin-releasing hormone (GnRH). Interaction between growth and reproduction occurs at various stages of the life cycle in fish. Depending on the species, growth-reproduction relationships can be contradictory, or be more or less dependent on environmental constraints. The objective is to maintain the fish at a growth rate or life stage that promotes accumulation of lipids, by controlling the timing of sexual maturation.

In one specific aspect of the invention, the method comprises administering a hormone antagonist to the fish, such that sexual maturation of the fish is delayed or prevented. Prevention of maturation is effectively sterilization of the fish prior to harvest. By delaying or preventing reproductive functions such as vitellogenesis or spermatogenesis, the growth rate of the fish can be maintained at an optimal level. This aspect of the invention is particularly applicable to those species of fish that slows its growth as it approaches sexual maturity. Other advantages are a reduction of energy intensive courtship/territorial behavior, reduction in variation of harvest size, and reduction of the risk of environmental impact from escapes of the specially bred or transgenic fish of the invention.

In another specific aspect, the method comprises administering a hormone or an agonist thereof to the fish, such that sexual maturation of the fish is accelerated. This aspect of the invention is applicable to those species of fish that accumulate lipids in certain organs, such as liver, ovary, testes, as well as unfertilized eggs, as they approach sexual maturity. By advancing the onset of puberty, the fish begins accumulate lipids at an earlier age, thereby reducing the time to harvest. Lutenizing hormone (LH), follicle-stimulating hormone (FSH), and gonadotropin-releasing hormone (GnRH), orthologs from another species, or their analogs and agonist can be used to accelerate sexual maturation in the fish of the invention.

In many teleosts, body growth rate during the first months of life is an important parameter influencing the age of first sexual maturity. For example, sexual maturity in tilapia is a function of age, size and environmental conditions. The Mozambique tilapia reaches sexual maturity at a smaller size and younger age than the Nile and Blue tilapias. Tilapia populations in large lakes mature at a later age and larger size than the same species raised in small farm ponds. Typically, the Nile tilapia matures at about 10 to 12 months and 350 to 500 grams in East African lakes. Under good growth conditions this same species will reach sexual maturity in farm ponds at an age of 5 to 6 months and 150 to 200 grams. Under good growing conditions in ponds, the Mozambique tilapia may reach sexual maturity in as little as 3 months of age, when they seldom weigh more than 60 to 100 grams. In poorly fertilized ponds sexually mature Mozambique tilapia may be as small as 15 grams. Thus, depending on the species and the environmental conditions, the timing of sexual maturation can be controlled to maximize body weight and lipid yield.

In many species of cultured finfish, females exhibit higher growth rates than males and attain larger sizes. In addition, in some species, males mature before reaching marketable size. Together, this results in a larger dispersion of sizes and an overall reduction in production. The objective is to only produce fish of the gender that shows the greatest growth (e.g., females in salmonids and cyprinids, and males in cichlids). In one aspect, the use of oestrogens for sex control resulting in monosex female fish population is contemplated. In another aspect, the use of oestrogens for sex control resulting in monosex male fish population is contemplated.

Sex control is typically achieved by exposing sexually undifferentiated fish to exogenous steroids in order to direct the process of sex differentiation towards the desired sex. Oestrogens have been applied to at least 56 different species, using 12 different natural or synthetic oestrogenic substances. 17α-methyltestosterone (an androgen) and estradiol-17β (an oestrogen) are the most preferred hormones for induction of masculinization and feminization, respectively. Other feminizing hormonal substances include oestrone, oestriol, diethylstilbestrol (DES), DES diphosphate, DES dipropionate, and 17α-ethynyloestradiol. Any techniques available for the administration of hormones to fish can be used in the methods of the invention. Preferred techniques include immersion in a static or recirculating bath, and dietary treatment. Immersion is suitable for those species in which the responsive period coincides during larval stages, while dietary treatments are more appropriate for species in which the responsive period coincides with external feeding. The following formula can be used to estimate the dose and duration of a dietary treatment: dose in mg per kg diet multiplied by the duration of the treatment in days equals 2500, e.g., 25 mg hormone/kg diet for 100 days, or 50 mg hormone/kg diet for 50 days, and so on. Techniques well known in the art for endocrine control of sexual maturation and differentiation are described in publications, such as Piferrer, “Endocrine sex control strategies for the feminization of teleost fish,” Aquaculture 197, Issues 1-4:229-281 (2001); Beardmore et al. “Monosex male production in finfish as exemplified by tilapia: applications, problems, and prospects,” Aquaculture 197, Issues 1-4:283-301 (2001); Zohar et al. “Endocrine manipulations of spawning in cultured fish: from hormones to genes,” Aquaculture 197, Issues 1-4: 99-136 (2001).

### 4.7 Biofuel Production

Any fish processing technologies and means known in the art can be applied to obtain lipids and hydrocarbons from the fishes. In one embodiment of the invention, the entire body of a fish is used in making biofuel. The entire fish is processed to extract lipids without separating the fish fillet from other parts of the fish which are regarded as fish waste in the seafood industry. In another embodiment, only certain part(s) of the fish are used, e.g., non-fillet parts of a fish, fish viscera, head, liver, guts, testes, and/or ovary. Prior to being processed, the fishes of the invention are not treated to prevent or remove off-flavor taste of the flesh. The treatment may include culturing the fishes for a period from one day up to two weeks in an enclosure that has a lower algae and/or bacteria count than the fish enclosure.

Described below is an example of a method for processing the fishes of the invention. The processing step involves heating the fishes to greater than about 70° C., 80° C., 90° C. or 100° C., typically by a steam cooker, which coagulates the protein, ruptures the fat deposits and liberates lipids and oil and physico-chemically bound water, and; grinding, pureeing and/or pressing the fish by a continuous press with rotating helical screws. The fishes can be subjected to gentle pressure cooking and pressing which use significantly less energy than that is required to obtain lipids from algae. The coagulate may alternatively be centrifuged. This step removes a large fraction of the liquids (press liquor) from the mass, which comprises an oily phase and an aqueous fraction (stickwater). The separation of press liquor can be carried out by centrifugation after the liquor has been heated to 90° C. to 95° C. Separation of stickwater from oil can be carried out in vertical disc centrifuges. The lipids in the oily phase (fish oil) may be polished by treating with hot water which extracts impurities from the lipids to form biofuel. To obtain fish meal, the separated water is evaporated to form a concentrate (fish solubles) which is combined with the solid residues, and then dried to solid form (presscake). The dried material may be grinded to a desired particle size. The fish meal typically comprises mostly proteins (up to 70%), ash, salt, carbohydrates, and oil (about 5-10%). The fish meal can be used as animal feed and/or as an alternative energy feedstock

The invention provides a biofuel feedstock or a biofuel comprising lipids, hydrocarbons, or both, derived from fish that harvested algae according to the methods of the invention. Lipids of the invention can be subdivided according to polarity: neutral lipids and polar lipids. The major neutral lipids are triglycerides, and free saturated and unsaturated fatty acids. The major polar lipids are acyl lipids, such as glycolipids and phospholipids. A composition comprising lipids and hydrocarbons of the invention can be described and distinguished by the types and relative amounts of key fatty acids and/or hydrocarbons present in the composition.

A great variety of unsaturated or polyunsaturated fatty acids are produced by fish mostly with C₁₂ to C₂₂ carbon chains and 1 to 6 double bonds, mainly in cis configurations (Stansby, M. E., “Fish oils,” The Avi Publishing Company, Westport, Conn., 1967). Fish oil comprises about 90% triglycerides, about 5-10% monoglycerides and diglycerides, and about 1-2% sterols, glyceryl ethers, hydrocarbons, and fatty alcohols. One of skill would understand that the amount and variety of lipids in fish oil varies from one fish species to another, and also with the season of the year, the algae diet, spawning state, and environmental conditions. Fatty acids produced by the fishes of the invention comprise, without limitation, one or more of the following: 12:0, 14:0, 14:1, 15:branched, 15:0, 16:0, 16:1, 16:2 n-7, 16:2 n-4, 16:3 n-4, 16:3 n-3, 16:4 n-4, 16:4 n-1, 17:branched, 17:0, 17:1, 18:branched, 18:0, 18:1, 18:2 n-9, 18:2 n-6, 18:2 n-4, 18:3 n-6, 18:3 n-6, 18:3 n-3, 18:4 n-3, 19:branched, 19:0, 19:1, 20:0, 20:1, 20:2 n-9, 20:2 n-6, 20:3 n-6, 20:3 n-3, 20:4 n-6, 20:4 n-3, 20:5 n-3, 21:0, 21:5 n-2, 22:0, 22:1 n-11, 22:2, 22:3 n-3, 22:4 n-3, 22:5 n-3, 22:6 n-3, 23:0, 24:0, 24:1 (where n is the first double bond counted from the methyl group). See, also Jean Guillaume, Sadisivam Kaushik, Pierre Bergot, and Robert Metailler, “Nutrition and Feeding of Fish and Crustaceans,” Springer-Praxis, UK, 2001). In various embodiments of the invention, the improved fish may produce new lipid(s) that are normally not produced or produced only in negligible amounts in the unimproved fish. It is also expected that in many embodiments of the invention, the relative abundance of different lipids in an improved fish is changed relative to an unimproved fish.

In various embodiments, the invention also encompasses methods of making a liquid fuel which comprise processing lipids derived from fish that harvested algae. Products of the invention made by the processing of fish-derived biofuel feedstocks can be incorporated or used in a variety of liquid fuels including but not limited to, diesel, biodiesel, kerosene, jet-fuel, gasoline, JP-1, JP-4, JP-5, JP-6, JP-7, JP-8, Jet Propellant Thermally Stable (JPTS), Fischer-Tropsch liquids, alcohol-based fuels including ethanol-containing transportation fuels, other biomass-based liquid fuels including cellulosic biomass-based transportation fuels. Triacylglycerides in fish oil can also be converted to fatty acid methyl esters (FAME or biodiesel) by base-catalyzed transesterification, acid-catalyzed transesterification, enzyme-catalyzed transesterification, or supercritical methanol transesterification.

Non-limiting examples of systems and methods for processing lipids such as fish lipids into biofuel, can be found in the following patent publications, the entire contents of each of which are incorporated by reference herein: U.S Patent Publication No. 2007/0010682, entitled “Process for the Manufacture of Diesel Range Hydrocarbons;” U.S. Patent Publication No. 2007/0131579, entitled “Process for Producing a Saturated Hydrocarbon Component;” U.S. Patent Publication No. 2007/0135316, entitled “Process for Producing a Saturated Hydrocarbon Component;” U.S. Patent Publication No. 2007/0135663, entitled “Base Oil;” U.S. Patent Publication No. 2007/0135666, entitled “Process for Producing a Branched Hydrocarbon Component;” U.S. Patent Publication No. 2007/0135669, entitled “Process for Producing a Hydrocarbon Component;” and U.S. Patent Publication No. 2007/0299291, entitled “Process for the Manufacture of Base Oil.”

### 5. EXAMPLE

The present invention may be better understood by reference to the following non-limiting example, which is provided only as exemplary of the invention. The example should in no way be construed as limiting the broader scope of the invention.

In this example, a transgenic carp that feeds on microalgae and has a lipid content higher than wild type carp is produced by overexpressing ectopically a homologous melanocorptin antagonist—the agouti-related protein (AgRP) of goldfish. The transgenic fish can be used to harvest algae and produce biofuel according to the methods of the invention.

Common carp (Cyprinus carpio) is a widespread freshwater fish that is farmed worldwide, especially in China where it is accountable for a high percentage of the annual tonnage of the country's aquaculture output. It is very closely related to the common goldfish (Carassius auratus), with which it is capable of interbreeding (Taylor, J., R. Mahon. 1977. Hybridization of Cyprinus carpio and Carassius auratus, the first two exotic species in the lower Laurentian Great Lakes. Environmental Biology Of Fishes 1(2):205-208). The common carp, gold fish, and zebrafish all belong to the taxonomic family of Cyprinidae.

Agouti-related protein (AgRP) is a naturally occurring antagonist of melanocortin which plays a key role in the control of energy balance by antagonizing melanocortin effects at melanocortin 4 receptors in mammals. Blockade of the melanocortin system causes a distinct obesity syndrome in mice and humans. The AgRP-encoding cDNA was first cloned in mice and human by similarity screening of expressing sequence tags based on the pattern of cysteine in the C-terminal region of agouti. AgRP protein lacks the highly basic N-terminal and proline-rich regions, but it shares strong homology to agouti protein within the polycysteine domain (1997, Ollmann et al., Antagonism of central melanocortin receptors in vitro and in vivo by agouti-related protein. Science 278:135-138). AgRP contains 10 cysteine residues, nine of them spatially conserved, that form five disulfide bridges essential for the conformational stability and biological functions. By homology screening of a goldfish genomic library, the nucleotide and deduced amino acid sequence of goldfish AgRP was determined, and shown to expressed centrally and peripherally. (2003, Cerdá-Reverter et al., Endogenous Melanocortin Antagonist in Fish: Structure, Brain Mapping, and Regulation by Fasting of the Goldfish Agouti-Related Protein Gene, Endocrinology Vol. 144, No. 10 4552-4561). The protein has 128 amino acid residues and the amino acid sequence has been deposited at Genebank as Locus CAD88211 (GI: 36788207). The corresponding 651-bp mRNA sequence has been deposited at Genbank as Locus AJ555492 (GI: 36788206). Zebrafish AgRP cDNA encodes a 127-amino-acid protein 36% and 40% identical to human and mouse AGRP, respectively. The nucleotide sequences for AgRP homologs of salmon (Salmo salar) and puffer fish (Takifugu rubripes) are also available in sequence databases (GeneID: 100286779, 10028678, 100049636, 100049637).

A DNA fragment comprising a nucleotide sequence that encodes the goldfish AgRP and polylinker cloning sites at both ends is made by commercial DNA synthesis and assembled by polymerase chain reaction (PCR). A transgene comprising the goldfish AgRP DNA fragment positioned for expression driven by a β-actin promoter is constructed by recombinant DNA techniques, and propagated as a plasmid based on the pcDNA3.1+ vector (Invitrogen). The β-actin gene sequence of common carp has been deposited at Genebank Locus CYIACTBA accession M24113 and its promoter has been used to create a transgenic carp overexpressing a synthetic growth hormone (2008, Guan et al., Metabolism traits of ‘all fish’ growth hormone transgenic common carp (Cyprinus carpio L.) Aquaculture 284 (208) 217-223). Orientation and joined sequences are all verified by DNA sequencing.

To remove RNA contamination of the transgene construct, plasmid DNA is gel purified and resuspended at 100 ng/ul. About 1 nl of DNA is microinjected by pulled glass micropipette into each fertilized egg. Fertilized eggs of common carp are prepared by natural mating of non-transgenic parents under conventional aquaculture conditions. Microinjected embryos are raised to the adult stage on microalgae in tanks and field ponds. F0 founders capable of germline transmission are identified by amplification of genomic DNA by PCR using primers that hybridize to the transgene construct, and breeding. Genomic DNA of FO male is obtained from sperm. Transgene-positive males and females are separated and crossed with wild types. F1 transgenic carps are produced by crossing naturally a transgenic male F0 with a non-transgenic female. F2 and F3 transgenic carp are produced by natural matings and similarly genotyped by PCR.

Adult wild type, F2, and F3 transgenic fishes are randomly housed in a cluster of field ponds containing microalgae, and cultured for at least 20 days and up to 3 months in comparable density. The total number of fish is about 600, with about 200 wild type fish as control, about 200 F2 transgenic fish and about 200 F3 transgenic fish. Conventional methods of culturing common carps are followed (1985, A Hatchery Manual for the Common, Chinese, and Indian major carps, Jingran V. G. and Pullin R. S. V., published by International Centre for Living Aquatic Resources Management, Asian Development Bank, which is incorporated herein by reference in its entirety). At the end of the experiment, the fishes are sacrificed, measured lengthwise and depth-wise, weighed, and genotyped. Comparisons of energy contents, lipid contents, protein contents of control and transgenic fish are made while matching the gender, length and/or weight of the fishes. The growth rate of the fishes are also compared by sampling regularly during the culture period. Energy content is determined by bomb calorimetry using fish carcasses that are steamed and then dried at 70° C.

Lipid content and quality are determined by using a sample of a homogenate of an entire fish or oil extracted from steamed and pressed fish carcasses, and subjecting the sample to extraction by the Bligh-Dyer salt technique, which is followed by chromatographic separation and detection of fatty acids and triglycerides. Triglycerides are converted to fatty acid methyl esters (FAME) by base-catalyzed transesterification using methanol and sodium methoxide. The crude FAME is then washed with distilled water to remove impurities. Three distillations are conducted on the resulting FAMEs by a thin film/short path vacuum distillation apparatus (Pope Scientific). Triglyceride, fatty acid, and/or FAME profiles of different samples are used for comparisons.

Distribution of lipids in the body and obesity of the fishes are studied by analysis of adipocytes in paraffin-embedded sections of parts of the fishes, i.e., viscera, muscles, liver, body wall (for subcutaneous locations). The sections are stained with hematoxylin and eosin, and imaged digitally. The images are analyzed by the NIH Image J program (2007, Collins T J. “ImageJ for microscopy,” BioTechniques 43 (1 Suppl): 25-30) to determine adipocyte size and adipocyte density in randomly chosen grids within an image.

Since the common carp and goldfish are closely related and can interbreed naturally, the goldfish AgRP protein is expected to be functional when expressed in the common carp. Use of the β-actin promoter of the common carp ensures constitutive expression of the goldfish AgRP in most tissues of the transgenic fish. The transgenic carps (F2 and F3) are on average heavier and longer than the control fish of the same age due presumably to an increased rate of weight gain and linear growth. The total lipid content of the transgenic fish, as exemplified by triglyceride content, is expected to be higher than a control fish of the same age, and even if the transgenic and control fishes are of similar weight and/or length. The higher lipid content of the transgenic carp can be partially accounted for by an increase in adipocyte number and cell size as observed in histological sections of the transgenic fish. These transgenic fish that are farmed in open ponds and fed on microalgae can be employed to harvest microalgae efficiently and then used to produce biofuel according to the invention.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed:

1. A method for producing a fish, comprising:
(i) introducing a transgene into a fish, wherein the presence of said transgene results in a modification of the lipid content of said fish, and
(ii) selecting a fish or a progeny thereof that comprises said transgene, wherein the lipid content of said fish or progeny thereof is greater than the lipid content of a fish without said transgene.

2. A method for producing a fish, comprising:
(i) reproducing a population of fish according to a breeding program that is directed to modifying a phenotype, wherein said phenotype is lipid content, and
(ii) selecting a fish from a succeeding generation in the breeding program, wherein the lipid content of said fish is greater than the lipid content of fish of an earlier generation.

3. A method for culturing fish, comprising:
administering an antagonist of a fish hormone to a fish to prevent sexual maturation of the fish, wherein the fish hormone is lutenizing hormone, follicle stimulating hormone, or gonadotropin releasing hormone, and wherein the growth rate of a sexually mature fish is lower than the growth rate of a sexually immature fish, thereby increasing the lipid content of the fish.

4. A method for culturing fish, comprising:
administering a fish hormone or an agonist thereof to a fish to accelerate sexual maturation of a female fish, wherein the fish hormone is lutenizing hormone, follicle stimulating hormone, or gonadotropin releasing hormone, and wherein the lipid content of a sexually mature female fish is greater than the lipid content of a sexually immature female fish.

5. A fish comprising a transgene wherein the presence of said transgene results in a lipid content higher than a fish without said transgene, or a change in the quality and quantity of lipids relative to a fish without said transgene.

6. A fish produced by a method that comprises:
(i) reproducing a population of fish according to a breeding program that is directed to modifying a phenotype, wherein said phenotype is lipid content or lipid content and quality, and
(ii) selecting a fish from a succeeding generation in the breeding program, wherein the lipid content of said fish is greater than the lipid content of fish of an earlier generation.

7. A method for producing biofuel, comprising:
(i) providing a fish of claim 5 or 6,
(ii) feeding the fish with algae for a period of time,
(iii) extracting oil from the fish, and
(iv) converting the oil to biofuel.

8. The method of claim 1 or 2, wherein said selecting step comprises feeding said fish with algae from an algal culture of defined composition for a period of time, prior to determining the lipid content of said fish.

9. The method of claim 3 or 4, further comprising feeding said fish with algae from an algal culture of defined composition.

10. The method of claim 1 or 2, wherein the lipid content of said fish is estimated by determining the moisture content of said fish or a part or organ of said fish.

11. The method of claim 1, wherein said transgene comprises: (i) an expressible stimulator gene; or (ii) a gene expression regulatory region that is integrated into the genome and is operably associated with a native stimulator gene such that the stimulator gene is expressed ectopically or constitutively.

12. The method of claim 1, wherein said transgene comprises: (i) an expressible antisense polynucleotide of a suppressor gene; (ii) an expressible polynucleotide that silences expression of a suppressor gene by RNA interference; or (iii) an non-expressing allele of a suppressor gene that is integrated into the native suppressor gene in the genome.

13. The method of claim 1, wherein the stimulator gene encodes neuropeptide Y, pancreatic peptide, agouti-related protein (AgRP), a secretin, ghrelin, insulin, an insulin-like growth factor, orexin A, orexin B, galanin, a receptor of one of the foregoing factors, PPARγ, lipoprotein lipase, fat-induced transcript 1, or fat-induced transcript 2.

14. The method of claim 1, wherein the suppressor gene encodes leptin, cholecystokinin, cocaine and amphetamine-regulated transcript, corticotropin-releasing factor, bombesin, alpha-melanocyte-stimulating hormone, tachykinin, glucagon-like peptide-1, urotensin I, somatostatin, a receptor of one of the foregoing factors, PPARα, PPARδ, β-glucocerebrosidase, α-galactosidase, β-N-acetylhexosaminidase A, acid sphingomyelinases, NPC1, or NPC2.

15. The method of claim 2, wherein said breeding program comprises at least one of inbreeding, selective breeding, crossbreeding, induction of polyploidy, gynogenesis or androgenesis.

16. The method of claim 1, 2, 3, or 4, wherein said fish is a planktivore or an omnivore.

17. The method of claim 1, 2, 3 or 4, wherein said fish is a member of Clupiformes.

18. The method of claim 1, 2, 3 or 4, wherein said fish is a menhaden, shad, herring, sardine, hilsa, anchovy, milkfish, catfish, barb, carp, zebrafish, goldfish, loach, shiner, minnow, rasbora, Labeo species, smelt, or mullet.

19. The fish of claim 5 or 6, which is a member of Clupiformes.

20. The fish of claim 5 or 6, which is a menhaden, shad, herring, sardine, hilsa, anchovy, milkfish, catfish, barb, carp, zebrafish, goldfish, loach, shiner, minnow, rasbora, Labeo species, smelt, or mullet.

21. The method of claim 7, wherein the fish is a member of Clupiformes.

22. The method of claim 7, wherein the fish is in a monosex population.

23. The method of claim 7, wherein the fish is a menhaden, shad, herring, sardine, hilsa, anchovy, milkfish, catfish, barb, carp, zebrafish, goldfish, loach, shiner, minnow, rasbora, Labeo species, smelt, or mullet.

24. The method of claim 7, wherein the transgene encodes an agouti-related protein.

25. The method of claim 24, wherein the fish is common carp and the transgene encodes an agouti-related protein of goldfish.
